(19) United States
(12) Patent Application Publication
(10) Pub. No.: US 2006/0004056 A1
(43) Pub. Date: Jan. 5, 2006

(54) Di-aryl-substituted-ethan pyridone pde4 inhibitors

(76) Inventors: Bernard Cote, Quebec (CA); Evelyn Martins, Quebec (CA)

Correspondence Address: MERCK AND CO., INC, P O BOX 2000, RAHWAY, NJ, 07065-0907, US

(21) Appl. No.: 10/518,294

(22) PCT Filed: Jul. 2, 2003
(86) PCT No.: PCT/CA03/00995

§ 371 Date: Aug. 1, 2005

Related U.S. Application Data
(60) Provisional application No. 60/393,281, filed on Jul. 2, 2002.

(51) Int. Cl. A61K 31/444; A61K 31/4439; C07D 401/02; C07D 417/02

(52) U.S. Cl. 514/332; 514/340; 546/250; 546/269.7

(57) ABSTRACT

Di-aryl substituted ethane pyridones, particularly ethane pyridones substituted with i) a phenyl, ii) a pyridyl, iii) a thiazole, iv) a pyrimidinyl, v) a pyridazinyl, vi) a furyl, vii) a thienyl, viii) an oxazolyl, ix) an isoxazolyl, or x) an isothiazolyl moiety are phosphodiesterase-4 inhibitors useful in the treatment or prevention of asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; ankylosing spondylitis; osteoarthritis; inflammation; or cytokine-mediated chronic tissue degeneration.

### BACKGROUND OF THE INVENTION

1. Field of the Invention

The present invention is directed to compounds that are di-aryl substituted ethane pyridones. In particular, this invention is directed to ethane pyridones substituted with i) a phenyl, ii) a pyridyl, iii) a thiazole, iv) a pyrimidinyl, v) a pyridazinyl, vi) a furyl, vii) a thienyl, viii) an oxazolyl, ix) an isoxazolyl, or x) an isothiazolyl moiety which are phosphodiesterase-4 inhibitors.

2. Related Background

Hormones are compounds that variously affect cellular activity. In many respects, hormones act as messengers to trigger specific cellular responses and activities. Many effects produced by hormones, however, are not caused by the singular effect of just the hormone. Instead, the hormone first binds to a receptor, thereby triggering the release of a second compound that goes on to affect the cellular activity. In this scenario, the hormone is known as the first messenger while the second compound is called the second messenger. Cyclic adenosine monophosphate (adenosine 3′,5′-cyclic monophosphate, “cAMP” or “cyclic AMP”) is known as a second messenger for hormones including epinephrine, glucagon, calcitonin, corticotrophin, lipotropin, luteinizing hormone, norepinephrine, parathyroid hormone, thyroid-stimulating hormone, and vasopressin. Thus, cAMP mediates cellular responses to hormones. Cyclic AMP also mediates cellular responses to various neurotransmitters.

Phosphodiesterases (“PDE”) are a family of enzymes that metabolize 3′, 5′ cyclic nucleotides to 5′ nucleoside monophosphates, thereby terminating cAMP second messenger activity. A particular phosphodiesterase, phosphodiesterase-4 (“PDE4”, also known as “PDE-IV”), which is a high affinity, cAMP specific, type IV PDE, has generated interest as potential targets for the development of novel anti-asthmatic and anti-inflammatory compounds. PDE4 is known to exist as at lease four isoenzymes, each of which is encoded by a distinct gene. Each of the four known PDE4 gene products is believed to play varying roles in allergic and/or inflammatory responses. Thus, it is believed that inhibition of PDE4, particularly the specific PDE4 isoforms that produce detrimental responses, can beneficially affect allergy and inflammation symptoms. It would be desirable to provide novel compounds and compositions that inhibit PDE4 activity.

Inhibition of PDE4 activity is believed effective for the treatment of osteoporosis by reducing bone loss. For example, Ken-ici Miyamoto et al., Biochem. Pharmacology, 54:613-617(1997) describes the effect of a PDE4 on bone loss. Therefore, it would be desirable to provide novel compounds and compositions that inhibit PDE4 activity.

A major concern with the use of PDE4 inhibitors is the side effect of emesis which has been observed for several candidate compounds as described in C. Burnouf et al., (“Burnouf”), Ann. Rep. In Med. Chem., 33:91-109(1998). B. Hughes et al., Br. J. Pharmacol., 118:1183-1191(1996); M. J. Perry et al., Cell Biochem. Biophys., 29:113-132(1998); S. B. Christensen et al., J. Med. Chem., 41:821-835(1998); and Burnouf describe the wide variation of the severity of the undesirable side effects exhibited by various compounds. As described in M. D. Houslay et al., Adv. In Pharmacol., 44:225-342(1998) and D. Spina et al., Adv. In Pharmacol., 44:33-89(1998), there is great interest and research of therapeutic PDE4 inhibitors.

U.S. Pat. Nos. 5,622,977, 5,710,160, 5,710,170, 5,798,373, 5,849,770, and International Patent Publication No. WO 99/50262 describe tri-substituted aryl derivative PDE IV inhibitors, including tri-aryl ethane derivatives.

Compounds that include ringed systems are described by various investigators as effective for a variety of therapies and utilities. For example, International Patent Publication No. WO 98/25883 describes ketobenzamides as calpain inhibitors, European Patent Publication No. EP 811610 and U.S. Pat. Nos. 5,679,712, 5,693,672 and 5,747,541 describe substituted benzoylguanidine sodium channel blockers, U.S. Pat. No. 5,736,297 describes ring systems useful as a photosensitive composition. International Patent Publication WO9422852 describes quinolines as PDE4 inhibitors.

U.S. Pat. Nos. 5,491,147, 5,608,070, 5,739,144, 5,776,958, 5,780,477, 5,786,354, 5,859,034, 5,866,593, 5,891,896, and International Patent Publication WO 95/35283 describe PDE4 inhibitors that are tri-substituted aryl or heteroaryl phenyl derivatives. U.S. Pat. No. 5,580,888 describes PDE4 inhibitors that are styryl derivatives. U.S. Pat. No. 5,550,137 describes PDE4 inhibitors that are phenylaminocarbonyl derivatives. U.S. Pat. No. 5,340,827 describes PDE4 inhibitors that are phenylcarboxamide compounds. U.S. Pat. No. 5,780,478 describes PDE4 inhibitors that are tetra-substituted phenyl derivatives. International Patent Publication WO 96/00215 describes substituted oxime derivatives useful as PDE4 inhibitors. U.S. Pat. No. 5,633,257 describes PDE4 inhibitors that are cyclo(alkyl and alkenyl)phenyl-alkenyl (aryl and heteroaryl) compounds.

However, there remains a need for novel compounds and compositions that therapeutically inhibit PDE4 with minimal side effects.

### SUMMARY OF THE INVENTION

The present invention is directed to novel di-aryl substituted ethane pyridones. In particular, this invention is directed to ethanes substituted with i) a phenyl, ii) a pyridyl, iii) a thiazole, iv) a pyrimidinyl, v) a pyridazinyl, vi) a furyl, vii) a thienyl, viii) an oxazolyl, ix) an isoxazolyl, or x) an isothiazolyl moiety which are phosphodiesterase-4 inhibitors. This invention also provides a pharmaceutical composition which includes an effective amount of the novel di-aryl substituted ethane pyridone and a pharmaceutically acceptable carrier. This invention further provides a method of treatment in mammals of, for example, asthma, chronic bronchitis, chronic obstructive pulmonary disease (COPD), eosinophilic granuloma, psoriasis and other benign or malignant proliferative skin diseases, endotoxic shock (and associated conditions such as laminitis and colic in horses), septic shock, ulcerative colitis, Crohn's disease, reperfusion injury of the myocardium and brain, inflammatory arthritis, chronic glomerulonephritis, atopic dermatitis, urticaria, adult respiratory distress syndrome, infant respiratory distress syndrome, chronic obstructive pulmonary disease in animals, diabetes insipidus, allergic rhinitis, allergic conjunctivitis, vernal conjunctivitis, arterial restenosis, ortherosclerosis, atherosclerosis, neurogenic inflammation, pain, cough, rheumatoid arthritis, ankylosing spondylitis, transplant rejection and graft versus host disease, hypersecretion of gastric acid, bacterial, fungal or viral induced sepsis or septic shock, inflammation and cytokine-mediated chronic tissue degeneration, osteoarthritis, cancer, cachexia, muscle wasting, depression, memory impairment, tumour growth, cancerous invasion of normal tissues, osteoporosis, and bone loss by the administration of an effective amount of the novel ethane pyridone substituted with i) a phenyl, ii) a pyridyl, iii) a thiazole, iv) a pyrimidinyl, v) a pyridazinyl, vi) a furyl, vii) a thienyl, viii) an oxazolyl, ix) an isoxazolyl, or x) an isothiazolyl moiety which are phosphodiesterase-4 inhibitors.

### DETAILED DESCRIPTION OF THE INVENTION

A compound of this invention is represented by Formula (I): 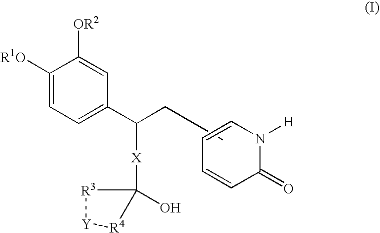
or a pharmaceutically acceptable salt thereof, wherein
X is phenyl, pyridinyl, thiazolyl, pyrimidinyl, pyridazinyl, furyl, thienyl, oxazolyl, isoxazolyl, isothiazolyl.

R¹ and R² are each independently —C₁₋₆alkyl, —C₃₋₆cycloalkyl, any of which optionally substituted with 1-6 independent halogen;
R³ and R⁴ are each independently —C₁₋₆alkyl, —C₃₋₆cycloalkyl, aryl, or heteroaryl, any of which optionally substituted with 1-6 independent halogen,
R³ and R⁴ are optionally connected by Y to form a ring, wherein Y is —C₁₋₁₆alkyl-.

In one aspect of this invention, a compound of this invention is represented by Formula (I), or a pharmaceutically acceptable salt thereof, wherein
X is phenyl, pyridinyl, or thiazolyl;
R¹ and R² are each independently —C₁₋₄alkyl, —C₃₋₆cycloalkyl, any of which optionally substituted with 1-6 independent halogen;
R³ and R⁴ are each independently —C₁₋₄alkyl optionally substituted with 1-6 independent halogen; and
R³ and R⁴ are optionally connected by Y to form a ring, wherein Y is —C₁₋₄alkyl-.

In one embodiment of this one aspect, the compounds of this invention are represented by Formula (I), or a pharmaceutically acceptable salt thereof, wherein
X is phenyl;
R¹ and R² are each independently —C₁₋₄alkyl, —C₃₋₆cycloalkyl, any of which optionally substituted with 1-6 independent halogen;
R³ and R⁴ are each independently —C₁₋₄alkyl optionally substituted with 1-6 independent halogen; and
R³ and R⁴ are optionally connected by Y to form a ring, wherein Y is —C₁₋₄alkyl-.

In an embodiment of this one aspect, the compounds of this invention are represented by Formula (I), or a pharmaceutically acceptable salt thereof, wherein
X is phenyl;
R¹ and R² are each independently —C₁₋₄alkyl, —C₃₋₆cycloalkyl, any of which optionally substituted with 1-6 independent halogen; and
R³ and R⁴ are each independently —C₁₋₄alkyl optionally substituted with 1-6 independent halogen.

In another embodiement of this one aspect, the compounds of this invention are represented by Formula (I), or a pharmaceutically acceptable salt thereof, wherein
X is phenyl;
R¹ and R² are each independently —C₁₋₄alkyl optionally substituted with 1-6 independent halogen; and
R³ and R⁴ are each independently —C₁₋₄alkyl optionally substituted with 1-6 independent halogen.

In a second aspect, the compounds of this invention are represented by Formula (I), or a pharmaceutically acceptable salt thereof, wherein
X is pyridinyl;
R¹ and R² are each independently —C₁₋₄alkyl, —C₃₋₆cycloalkyl, any of which optionally substituted with 1-6 independent halogen;
R³ and R⁴ are each independently —C₁₋₄alkyl optionally substituted with 1-6 independent halogen; and
R³ and R⁴ are optionally connected by Y to form a ring, wherein Y is —C₁₋₄alkyl-.

In an embodiment of the second aspect, the compounds of this invention are represented by Formula (I), or a pharmaceutically acceptable salt thereof, wherein
X is pyridinyl;
R¹ and R² are each independently —C₁₋₄alkyl, —C₃₋₆cycloalkyl, any of which optionally substituted with 1-6 independent halogen; and
R³ and R⁴ are each independently —C₁₋₄alkyl optionally substituted with 1-6 independent halogen.

In another embodiment of the second aspect, the compounds of this invention are represented by Formula (I), or a pharmaceutically acceptable salt thereof, wherein
X is pyridinyl;
R¹ and R² are each independently —C₁₋₄alkyl optionally substituted with 1-6 independent halogen; and
R³ and R⁴ are each independently —C₁₋₄alkyl optionally substituted with 1-6 independent halogen.

In a third aspect, the compounds of this invention are represented by Formula (I), or a pharmaceutically acceptable salt thereof, wherein
X is thiazolyl;
R¹ and R² are each independently —C₁₋₄alkyl, —C₃₋₆cycloalkyl, any of which optionally substituted with 1-6 independent halogen;
R³ and R⁴ are each independently —C₁₋₄alkyl optionally substituted with 1-6 independent halogen; and
R³ and R⁴ are optionally connected by Y to form a ring, wherein Y is —C₁₋₄alkyl-.

In an embodiment of the third aspect, the compounds of this invention are represented by Formula (I), or a pharmaceutically acceptable salt thereof, wherein
X is thiazolyl;
R¹ and R² are each independently —C₁₋₄alkyl, —C₃₋₆cycloalkyl, any of which optionally substituted with 1-6 independent halogen; and
R³ and R⁴ are each independently —C₁₋₄alkyl optionally substituted with 1-6 independent halogen.

As used herein, “alkyl” as well as other groups having the prefix “alk” such as, for example, alkoxy, alkanoyl, alkenyl, alkynyl and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains containing at least one unsaturated C—C bond.

The term “cycloalkyl” means carbocycles containing no heteroatoms, and includes mono-, bi- and tricyclic saturated carbocycles, as well as fused ring systems. Such fused ring systems can include one ring that is partially or fully unsaturated such as a benzene ring to form fused ring systems such as benzofused carbocycles. Cycloalkyl includes such fused ring systems as spirofused ring systems. Examples of cycloalkyl include cyclopropyl, cyclobutyl, cyclopentyl, cyclohexyl, decahydronaphthalenyl, adamantanyl, indanyl, indenyl, fluorenyl, 1,2,3,4-tetrahydronaphthalenyl and the like. Similarly, “cycloalkenyl” means carbocycles containing no heteroatoms and at least one non-aromatic C—C double bond, and include mono-, bi- and tricyclic partially saturated carbocycles, as well as benzofused cycloalkenes. Examples of cycloalkenyl include cyclohexenyl, indenyl, and the like.

The term “cycloalkyloxy” unless specifically stated otherwise includes a cycloalkyl group connected to the oxy connecting atom.

The term “alkoxy” unless specifically stated otherwise includes an alkyl group connected to the oxy connecting atom.

The term “aryl” unless specifically stated otherwise includes multiple ring systems as well as single ring systems such as, for example, phenyl or naphthyl.

The term “aryloxy” unless specifically stated otherwise includes multiple ring systems as well as single ring systems such as, for example, phenyl or naphthyl, connected through the oxy connecting atom to the connecting site.

The term “C₀-C₆alkyl” includes alkyls containing 6, 5, 4, 3, 2, 1, or no carbon atoms. An alkyl with no carbon atoms is a hydrogen atom substituent when the alkyl is a terminus moiety. An alkyl with no carbon atoms is a direct bond when the alkyl is a bridging moiety.

The term “hetero” unless specifically stated otherwise includes one or more O, S, or N atoms. For example, heterocycloalkyl and heteroaryl include ring systems that contain one or more O, S, or N atoms in the ring, including mixtures of such atoms. The heteroatoms replace ring carbon atoms. Thus, for example, a heterocycloC₅alkyl is a five membered ring containing from 5 to no carbon atoms.

Examples of heteroaryl include, for example, pyridinyl, quinolinyl, isoquinolinyl, pyridazinyl, pyrimidinyl, pyrazinyl, quinoxalinyl, furyl, benzofuryl, dibenzofuryl, thienyl, benzothienyl, pyrrolyl, indolyl, pyrazolyl, indazolyl, oxazolyl, isoxazolyl, thiazolyl, isothiazolyl, imidazolyl, benzimidazolyl, oxadiazolyl, thiadiazolyl, triazolyl, tetrazolyl.

The term “heteroaryloxy” unless specifically stated otherwise describes a heteroaryl group connected through an oxy connecting atom to the connecting site.

Examples of heteroaryl(C₁₋₆)alkyl include, for example, furylmethyl, furylethyl, thienylmethyl, thienylethyl, pyrazolylmethyl, oxazolylmethyl, oxazolylethyl, isoxazolylmethyl, thiazolylmethyl, thiazolylethyl, imidazolylmethyl, imidazolylethyl, benzimidazolylmethyl, oxadiazolylmethyl, oxadiazolylethyl, thiadiazolylmethyl, thiadiazolylethyl, triazolylmethyl, triazolylethyl, tetrazolylmethyl, tetrazolylethyl, pyridinylmethyl, pyridinylethyl, pyridazinylmethyl, pyrimidinylmethyl, pyrazinylmethyl, quinolinylmethyl, isoquinolinylmethyl and quinoxalinylmethyl.

Examples of heterocycloC₃₋₇alkyl include, for example, azetidinyl, pyrrolidinyl, piperidinyl, perhydroazepinyl, piperazinyl, morpholinyl, tetrahydrofuranyl, imidazolinyl, pyrolidin-2-one, piperidin-2-one, and thiomorpholinyl.

The term “N-heterocycloC₄₋₇alkyl” describes nonaryl heterocyclic compounds having 3-6 carbon atoms and one nitrogen atom forming the ring. Examples include azetidinyl, pyrrolidinyl, piperidinyl, and perhydroazepinyl.

Examples of aryl(C₁₋₆)alkyl include, for example, phenyl(C₁₋₆)alkyl, and naphthyl(C₁₋₆)alkyl.

Examples of heterocycloC₃₋₇alkylcarbonyl(C₁₋₆)alkyl include, for example, azetidinyl carbonyl(C₁₋₆)alkyl, pyrrolidinyl carbonyl(C₁₋₆)alkyl, piperidinyl carbonyl(C₁₋₆)alkyl, piperazinyl carbonyl(C₁₋₆)alkyl, morpholinyl carbonyl(C₁₋₆)alkyl, and thiomorpholinyl carbonyl(C₁₋₆)alkyl.

The term “amine” unless specifically stated otherwise includes primary, secondary and tertiary amines.

Unless otherwise stated, the term “carbamoyl” is used to include —NHC(O)OC₁-C₄alkyl, and —OC(O)NHC₁-C₄alkyl.

The term “halogen” includes fluorine, chlorine, bromine and iodine atoms.

The term “optionally substituted” is intended to include both substituted and unsubstituted. Thus, for example, optionally substituted aryl could represent a pentafluorophenyl or a phenyl ring. Further, the substitution can be made at any of the groups. For example, substituted aryl(C₁₋₆)alkyl includes substitution on the aryl group as well as substitution on the alkyl group.

The term “oxide” of heteroaryl groups is used in the ordinary well-known chemical sense and include, for example, N-oxides of nitrogen heteroatoms.

Compounds described herein contain one or more double bonds and may thus give rise to cis/trans isomers as well as other conformational isomers. The present invention includes all such possible isomers as well as mixtures of such isomers.

Compounds described herein can contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above Formula I is shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be mixtures of stereoisomers.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, manganese (ic and ous), potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include ion exchange resins such as, for example, arginine, betaine, caffeine, choline, N,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are benzenesulfonic, citric, hydrobromic, hydrochloric, maleic, phosphoric, sulfuric, and tartaric acids.

The pharmaceutical compositions of the present invention comprise a compound represented by Formula I (or pharmaceutically acceptable salts thereof) as an active ingredient, a pharmaceutically acceptable carrier and optionally other therapeutic ingredients or adjuvants. Such additional therapeutic ingredients include, for example, i) Leukotriene receptor antagonists, ii) Leukotriene biosynthesis inhibitors, iii) corticosteroids, iv) HI receptor antagonists, v) beta 2 adrenoceptor agonists, vi) COX-2 selective inhibitors, vii) statins, viii) non-steroidal anti-inflammatory drugs (“NSAID”), and ix) M2/M3 antagonists. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Creams, ointments, jellies, solutions, or suspensions containing the compound of Formula I can be employed for topical use. Mouth washes and gargles are included within the scope of topical use for the purposes of this invention.

Dosage levels from about 0.001 mg/kg to about 140 mg/kg of body weight per day are useful in the treatment of conditions such as i) Pulmonary disorders such as asthma, chronic bronchitis, chronic obstructive pulmonary disease (COPD), adult respiratory distress syndrome, infant respiratory distress syndrome, cough, chronic obstructive pulmonary disease in animals, adult respiratory distress syndrome, and infant respiratory distress syndrome, ii) Gastrointestinal disorders such as ulcerative colitis, Crohn's disease, and hypersecretion of gastric acid, iii) Infectious diseases such as bacterial, fungal or viral induced sepsis or septic shock, endotoxic shock (and associated conditions such as laminitis and colic in horses), and septic shock, iv) Neurological disorders such as spinal cord trauma, head injury, neurogenic inflammation, pain, and reperfusion injury of the brain, v) Inflammatory disorders such as psoriatic arthritis, rheumatoid arthritis, ankylosing spondylitis, osteoarthritis, inflammation and cytokine-mediated chronic tissue degeneration, vi) Allergic disorders such as allergic rhinitis, allergic conjunctivitis, and eosinophilic granuloma, vii) Psychiatric disorders such as depression, memory impairment, and monopolar depression, viii) Neurodegenerative disorders such as Parkinson disease, Alzheimer's disease, acute and chronic multiple sclerosis, ix) Dermatological disorders such as psoriasis and other benign or malignant proliferative skin diseases, atopic dermatitis, and urticaria, x) Oncological diseases such as cancer, tumor growth and cancerous invasion of normal tissues, xi) Metabolic disorders such as diabetes insipidus, xii) Bone disorders such as osteoporosis, xiii) Cardiovascular disorders such as arterial restenosis, atherosclerosis, reperfusion injury of the myocardium, and xiv) Other disorders such as chronic glomerulonephritis, vernal conjunctivitis, transplant rejection and graft versus host disease, and cachexia—which are responsive to PDE4 inhibition, or alternatively about 0.05 mg to about 7 g per patient per day. For example, inflammation may be effectively treated by the administration of from about 0.01 mg to 50 mg of the compound per kilogram of body weight per day, or alternatively about 0.5 mg to about 2.5 g per patient per day. Further, it is understood that the PDE4 inhibiting compounds of this invention can be administered at prophylactically effective dosage levels to prevent the above-recited conditions.

The amount of active ingredient that may be combined with the carrier materials to produce a single dosage form will vary depending upon the host treated and the particular mode of administration. For example, a formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms will generally contain between from about 0.01 mg to about 1000 mg of the active ingredient, typically 0.01 mg, 0.05 mg, 0.25 mg, 1 mg, 5 mg, 25 mg, 50 mg, 100 mg, 200 mg, 300 mg, 400 mg, 500 mg, 600 mg, 800 mg or 1000 mg.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

In practice, the compounds represented by Formula I, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, or pharmaceutically acceptable salts thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.1 mg to about 500 mg of the active ingredient and each cachet or capsule preferably containing from about 0.1 mg to about 500 mg of the active ingredient.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, or pharmaceutically acceptable salts thereof, may also be prepared in powder or liquid concentrate form.

The compounds and pharmaceutical compositions of this invention have been found to exhibit biological activity as PDE4 inhibitors. Accordingly, another aspect of the invention is the treatment in mammals of, for example, i) Pulmonary disorders such as asthma, chronic bronchitis, chronic obstructive pulmonary disease (COPD), adult respiratory distress syndrome, infant respiratory distress syndrome, cough, chronic obstructive pulmonary disease in animals, adult respiratory distress syndrome, and infant respiratory distress syndrome, ii) Gastrointestinal disorders such as ulcerative colitis, Crohn's disease, and hypersecretion of gastric acid, iii) Infectious diseases such as bacterial, fungal or viral induced sepsis or septic shock, endotoxic shock (and associated conditions such as laminitis and colic in horses), and septic shock, iv) Neurological disorders such as spinal cord trauma, head injury, neurogenic inflammation, pain, and reperfusion injury of the brain, v) Inflammatory disorders such as psoriatic arthritis, rheumatoid arthritis, ankylosing spondylitis, osteoarthritis, inflammation and cytokine-mediated chronic tissue degeneration, vi) Allergic disorders such as allergic rhinitis, allergic conjunctivitis, and eosinophilic granuloma, vii) Psychiatric disorders such as depression, memory impairment, and monopolar depression, viii) Neurodegenerative disorders such as Parkinson disease, Alzheimer's disease, acute and chronic multiple sclerosis, ix) Dermatological disorders such as psoriasis and other benign or malignant proliferative skin diseases, atopic dermatitis, and urticaria, x) Oncological diseases such as cancer, tumor growth and cancerous invasion of normal tissues, xi) Metabolic disorders such as diabetes insipidus, xii) Bone disorders such as osteoporosis, xiii) Cardiovascular disorders such as arterial restenosis, atherosclerosis, reperfusion injury of the myocardium, and xiv) Other disorders such as chronic glomerulonephritis, vernal conjunctivitis, transplant rejection and graft versus host disease, and cachexia—maladies that are amenable to amelioration through inhibition of the PDE4 isoenzyme and the resulting elevated cAMP levels—by the administration of an effective amount of the compounds of this invention. The term “mammals” includes humans, as well as other animals such as, for example, dogs, cats, horses, pigs, and cattle. Accordingly, it is understood that the treatment of mammals other than humans is the treatment of clinical correlating afflictions to those above recited examples that are human afflictions.

Further, as described above, the compound of this invention can be utilized in combination with other therapeutic compounds. In particular, the combinations of the PDE4 inhibiting compound of this invention can be advantageously used in combination with i) Leukotriene receptor antagonists, ii) Leukotriene biosynthesis inhibitors, iii) COX-2 selective inhibitors, iv) statins, v) NSAIDs, vi) M2/M3 antagonists, vii) corticosteroids, viii) H1 (histamine) receptor antagonists and ix) beta 2 adrenoceptor agonist.

Thus, for example, pulmonary disorders such as asthma, chronic bronchitis, chronic obstructive pulmonary disease (COPD), adult respiratory distress syndrome, infant respiratory distress syndrome, cough, chronic obstructive pulmonary disease in animals, adult respiratory distress syndrome, and infant respiratory distress syndrome can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Gastrointestinal disorders such as ulcerative colitis, Crohn's disease, and hypersecretion of gastric acid can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Infectious diseases such as bacterial, fungal or viral induced sepsis or septic shock, endotoxic shock (and associated conditions such as laminitis and colic in horses), and septic shock can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Neurological disorders such as spinal cord trauma, head injury, neurogenic inflammation, pain, and reperfusion injury of the brain can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Inflammatory disorders such as psoriatic arthritis, rheumatoid arthritis, ankylosing spondylitis, osteoarthritis, inflammation and cytokine-mediated chronic tissue degeneration can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Allergic disorders such as allergic rhinitis, allergic conjunctivitis, and eosinophilic granuloma can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Psychiatric disorders such as depression, memory impairment, and monopolar depression can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Neurodegenerative disorders such as Parkinson disease, Alzheimer's disease, acute and chronic multiple sclerosis can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Dermatological disorders such as psoriasis and other benign or malignant proliferative skin diseases, atopic dermatitis, and urticaria can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Oncological diseases such as cancer, tumor growth and cancerous invasion of normal tissues can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Metabolic disorders such as diabetes insipidus can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

Bone disorders such as osteoporosis, cardiovascular disorders such as arterial restenosis, atherosclerosis, reperfusion injury of the myocardium, and other disorders such as chronic glomerulonephritis, vernal conjunctivitis, transplant rejection and graft versus host disease, and cachexia can be conveniently treated with capsules, cachets or tablets each containing 1 mg, 5 mg, 25 mg, 50 mg, 100 mg, 200 mg, 300 mg, 400 mg, or 500 mg of the active ingredient of the compound of the present application, or a pharmaceutically acceptable salt thereof, administered once, twice, or three times daily.

The abbreviations used herein have the following tabulated meanings. Abbreviations not tabulated below have their meanings as commonly used unless specifically stated otherwise.

| | |
|---|---|
| Ac = | acetyl |
| Bn = | benzyl |
| CAMP | cyclic adenosine-3′,5′-monophosphate |
| DBU = | 1,8-diazabicyclo[5.4.0]undec-7-ene |
| DIBAL = | diisobutylaluminum hydride |
| DMAP = | 4-(dimethylamino)pyridine |
| DMF = | N,N-dimethylformamide |
| Et₃N = | triethylamine |
| GST | glutathione transferase |
| HMDS | hexamethyldisilazide |
| LDA = | lithium diisopropylamide |
| m-CPBA = | metachloroperbenzoic acid |
| MMPP = | monoperoxyphthalic acid |
| MPPM = | monoperoxyphthalic acid, magnesium salt 6H₂O |
| Ms = | methanesulfonyl = mesyl = SO₂Me |
| Ms0 = | methanesulfonate = mesylate |
| NSAID = | non-steroidal anti-inflammatory drug |
| o-Tol = | ortho-tolyl |
| OXONE ® = | 2KHSO₅.KHSO₄.K₂SO₄ |
| PCC = | pyridinium chlorochromate |
| PDC = | pyridinium dichromate |
| PDE | phosphodiesterase |
| Ph = | phenyl |
| Phe = | benzenediyl |
| PMB = | para-methoxybenzyl |
| Pye = | pyridinediyl |
| r.t. = | room temperature |
| Rac. = | racemic |
| SAM = | aminosulfonyl or sulfonamide or SO₂NH₂ |
| SEM = | 2-(trimethylsilyl)ethoxymethoxy |
| SPA = | scintillation proximity assay |
| TBAF = | tetra-n-butylammonium fluoride |
| Th = | 2- or 3-thienyl |
| TFA = | trifluoroacetic acid |
| TFAA = | trifluoroacetic acid anhydride |
| THF = | tetrahydrofuran |
| Thi = | thiophenediyl |
| TLC = | thin layer chromatography |
| TMS-CN = | trimethylsilyl cyanide |
| TMSI | trimethylsilyl iodide |
| Tz = | 1H (or 2H)-tetrazol-5-yl |
| CAN | ceric ammonium nitrate |
| C₃H₅ = | allyl |

Alkyl Group Abbreviations

| | |
|---|---|
| Me = | Methyl |
| Et = | ethyl |
| n-Pr = | normal propyl |
| i-Pr = | isopropyl |
| n-Bu = | normal butyl |
| i-Bu = | isobutyl |
| s-Bu = | secondary butyl |
| t-Bu = | tertiary butyl |
| c-Pr = | cyclopropyl |
| c-Bu = | Cyclobutyl |
| c-Pen = | cyclopentyl |
| c-Hex = | cyclohexyl |

### Assays Demonstrating Biological Activity

### LPS and FMLP-Induced TNF-α and LTB4 Assays in Human Whole Blood

Whole blood provides a protein and cell-rich milieu appropriate for the study of biochemical efficacy of anti-inflammatory compounds such as PDE4-selective inhibitors. Normal non-stimulated human blood does not contain detectable levels of TNF-α and LTB4. Upon stimulation with LPS, activated monocytes express and secrete TNF-α up to 8 hours and plasma levels remain stable for 24 hours. Published studies have shown that inhibition of TNF-α by increasing intracellular cAMP via PDE4 inhibition and/or enhanced adenylyl cyclase activity occurs at the transcriptional level. LTB₄ synthesis is also sensitive to levels of intracellular cAMP and can be completely inhibited by PDE4-selective inhibitors. As there is little LTB4 produced during a 24 hour LPS stimulation of whole blood, an additional LPS stimulation followed by fMLP challenge of human whole blood is necessary for LTB₄ synthesis by activated neutrophils. Thus, by using the same blood sample, it is possible to evaluate the potency of a compound on two surrogate markers of PDE4 activity in the whole blood by the following procedure.

Fresh blood was collected in heparinized tubes by venipuncture from healthy human volunteers (male and female). These subjects had no apparent inflammatory conditions and had not taken any NSAIDs for at least 4 days prior to blood collection. 500 μL aliquots of blood were pre-incubated with either 2 μL of vehicle (DMSO) or 2 μL of test compound at varying concentrations for 15 minutes at 37° C. This was followed by the addition of either 10 μL vehicle (PBS) as blanks or 10 μL LPS (1 μg/mL final concentration, #L-2630 (Sigma Chemical Co., St. Louis, Mo.) from E. coli, serotype 0111:B4; diluted in 0.1% w/v BSA (in PBS)). After 24 hours of incubation at 37° C., another 10 μL of PBS (blank) or 10 μL of LPS (1 μg/mL final concentration) was added to blood and incubated for 30 minutes at 37° C. The blood was then challenged with either 10 μL of PBS (blank) or 10 μL of fMLP (1 μM final concentration, #F-3506 (Sigma); diluted in 1% w/v BSA (in PBS)) for 15 minutes at 37° C. The blood samples were centrifuged at 1500×g for 10 minutes at 4° C. to obtain plasma. A 50 μL aliquot of plasma was mixed with 200 μL methanol for protein precipitation and centrifuged as above. The supernatant was assayed for LTB4 using an enzyme immunoassay kit (#520111 from Cayman Chemical Co., Ann Arbor, Mich.) according to the manufacturer's procedure. TNF-α was assayed in diluted plasma (in PBS) using an ELISA kit (Cistron Biotechnology, Pine Brook, N.J.) according to manufacturer's procedure. IC₅₀ values should be less than about 5 μM, advantageously less than about 2.5 μM.

### Anti-Allergic Activity In Vivo

Compounds of the invention have been tested for effects on an IgE-mediated allergic pulmonary inflammation induced by inhalation of antigen by sensitized guinea pigs. Guinea pigs were initially sensitized to ovalbumin under mild cyclophosphamide-induced immunosuppression, by intraperitoneal injection of antigen in combinations with aluminum hydroxide and pertussis vaccine. Booster doses of antigen were given two and four weeks later. At six weeks, animals were challenged with aerosolized ovalbumin while under cover of an intraperitoneally administered anti-histamine agent (mepyramine). After a further 48 h, bronchial alveolar lavages (BAL) were performed and the numbers of eosinophils and other leukocytes in the BAL fluids were counted. The lungs were also removed for histological examination for inflammatory damage. Administration of compounds of the Examples (0.001-10 mg/kg i.p. or p.o.), up to three times during the 48 h following antigen challenge, lead to a significant reduction in the eosinophilia and the accumulation of other inflammatory leukocytes.

### SPA Based PDE Activity Assay Protocol

Compounds which inhibit the hydrolysis of cAMP to AMP by the type-IV cAMP-specific phosphodiesterases were screened in a 96-well plate format as follows:

In a 96 well-plate at 30° C. the test compound was added (dissolved in 2 μL DMSO), 188 μL of substrate buffer containing [2,8-³H] adenosine 3′,5′-cyclic phosphate (cAMP, 100 nM to 50 μM), 10 mM MgCl₂, 1 mM EDTA, 50 mM Tris, pH 7.5. The reaction was initiated by the addition of human recombinant PDE4 (the amount was controlled so that ˜10% product was formed in 10 min.). The reaction was stopped after 10 min. by the addition of 1 mg of PDE-SPA beads (Amersham Pharmacia Biotech, Inc., Piscataway, N.J.). The product AMP generated was quantified on a Wallac Microbeta® 96-well plate counter (EG&G Wallac Co., Gaithersburg, Md.). The signal in the absence of enzyme was defined as the background. 100% activity was defined as the signal detected in the presence of enzyme and DMSO with the background subtracted. Percentage of inhibition was calculated accordingly. IC₅₀ value was approximated with a non-linear regression fit using the standard 4-parameter/multiple binding sites equation from a ten point titration.

The IC₅₀ values of Examples 1 to 9 were determined with 100 nM cAMP using the purified GST fusion protein of the human recombinant phosphodiesterase IVa (met-248) produced from a baculovirus/Sf-9 expression system. IC₅₀ values should be less than about 1000 nM, advantageously less than about 250 nM, and even more advantageously less than about 100 nM. The IC₅₀ values of Examples 1 to 9 ranged from 0.05 nM to 200 nM.

The Examples that follow are intended as an illustration of certain preferred embodiments of the invention and no limitation of the invention is implied.

Unless specifically stated otherwise, the experimental procedures were performed under the following conditions. All operations were carried out at room or ambient temperature—that is, at a temperature in the range of 18-25° C. Evaporation of solvent was carried out using a rotary evaporator under reduced pressure (600-4000 pascals: 4.5-30 mm Hg) with a bath temperature of up to 60° C. The course of reactions was followed by thin layer chromatography (TLC) and reaction times are given for illustration only. Melting points are uncorrected and “d” indicates decomposition. The melting points given are those obtained for the materials prepared as described. Polymorphism may result in isolation of materials with different melting points in some preparations. The structure and purity of all final products were assured by at least one of the following techniques: TLC, mass spectrometry, nuclear magnetic resonance (NMR) spectrometry or microanalytical data. When given, yields are for illustration only. When given, NMR data is in the form of delta (δ) values for major diagnostic protons, given in parts per million (ppm) relative to tetramethylsilane (TMS) as internal standard, determined at 300 MHz, 400 MHz or 500 MHz using the indicated solvent. Conventional abbreviations used for signal shape are: s. singlet; d. doublet; t. triplet; m. multiplet; br. broad; etc. In addition, “Ar” signifies an aromatic signal. Chemical symbols have their usual meanings; the following abbreviations have also been used: v (volume), w (weight), b.p. (boiling point), m.p. (melting point), L (liter(s)), mL (milliliters), g (gram(s)), mg (milligrams(s)), mol (moles), mmol (millimoles), eq (equivalent(s)).

### Methods of Synthesis

The compounds of Formula (I) of the present invention can be prepared according to the synthetic routes outlined in Schemes 1 and 2 below and by following the methods described therein. It is obvious to one skilled in the art that resolution of compounds bearing stereogenic centers, such as VIIb, Ia or XI for example, or compounds of Formula I, can be accomplished by one of several methods, including HPLC with a chiral column, or formation and crystallization of a salt prepared by reaction of the compound with a chiral acid or base. The substituents are the same as in Formula (I) except where defined otherwise.

Scheme 1

The pyridone of Formula Ia, lb and Ic may be prepared in a multi-step sequence from the requisite dialkoxyaldehyde III and an appropriately substituted bromophenyl Ia, bromopyridine IIb or thiazole IIc as presented in SCHEME 1 below. Addition of a metalated intermediate II, prepared by transmetallation of Ia or IIb or, by regioselective metalation of thiazole IIc with a base such as n-butyllithium in a suitable solvent such as ether or THF, to III provides secondary alcohol IV. Conversion of IV into the corresponding secondary chloride or bromide V is accomplished by reaction with an appropriate halogenating reagent, such as thionyl chloride or thionyl bromide, and an organic base, such as pyridine, diisopropylethylamine or triethylamine, in an organic solvent such as dichloromethane or toluene. Alkylation of the anion derived from deprotonation of an alkyl pyridylacetate or of an alkyl pyridylacetate-N-oxide with an appropriate base, such as lithium, sodium or potassium bis(trimethylsilyl)amide, with the halide V in an appropriate organic solvent such as THF and/or HMPA (hexamethylphosphoramide), provides the ester VI. Ester VI is decarboxylated to give the pyridine or the pyridine-N-oxide VII by first hydrolysing the ester VI in the presence of aqueous hydroxide, such as sodium hydroxide, in a mixture of protic and aprotic organic solvents, such as methanol or ethanol and THF, followed by heating the carboxylic acid in an organic solvent such as dimethylsulfoxide which also cause cleavage of the alcohol protecting group. Incomple deprotection would therefore lead to an additional separated step of alcohol deprotection with a suitable reagent such as trifluoroacetic acid of tetrabutylammonium fluoride in an organic solvent such as methylene chloride or THF. Reaction of VIIa, VIIb or VIIc₁ with an oxidizing agent, such as m-CPBA (meta-chloroperoxybenzoic acid) or MMPP (monoperoxyphthalic acid, magnesium salt) provides the N-oxides VIIa₂, VIIb₂ or VIIc₂. These pyridine-N-oxide are rearranged to pyridone of Formula Ia, lb and Ic by heating the N-oxide in the presence of an anhydride such as trifluoroacetic anhydride or acetic anhydride and alternatively by treatment with the same anhydrides with an organic base such as pyridine, diisopropylethylamine or triethylamine in an organic solvent such as THF or DMF. 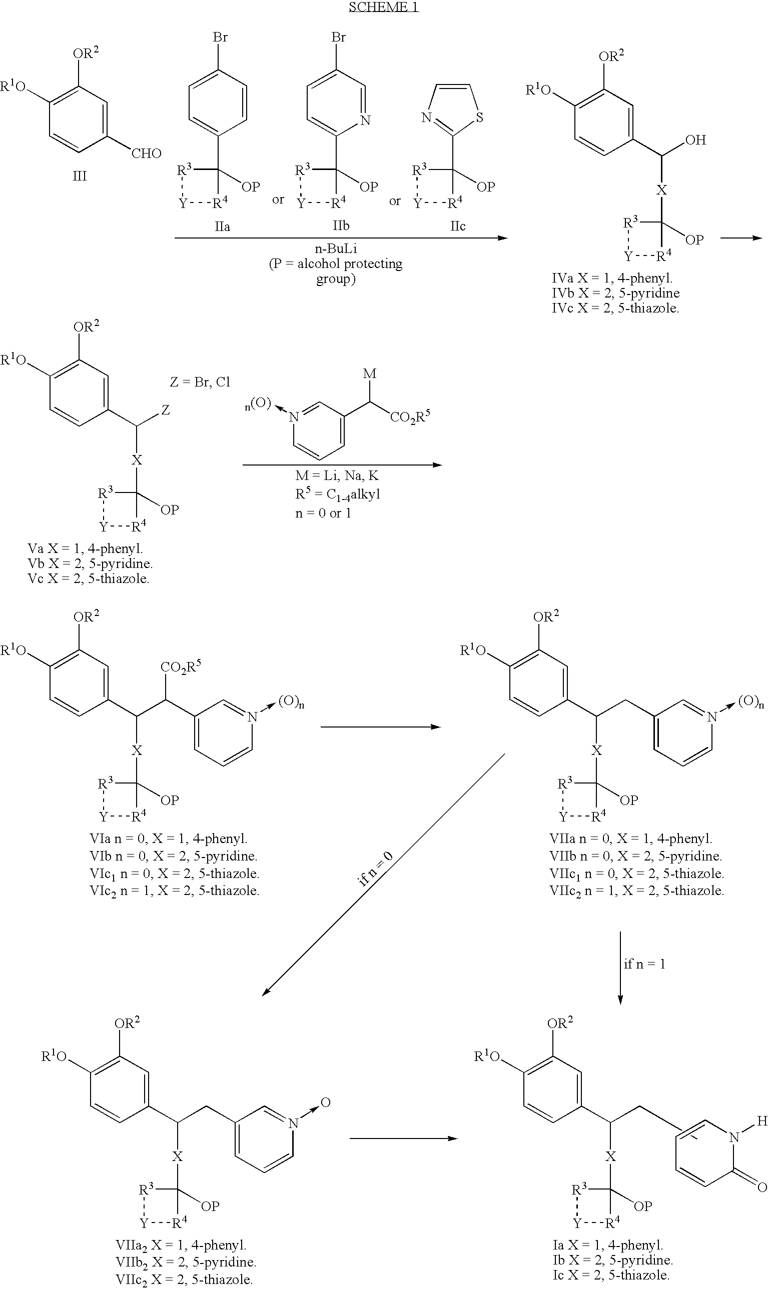
Scheme 2

The pyridyl pyridone of Formula Ib may also be prepared in a multi-step sequence from the requisite dialkoxyaldehyde III and 2,5-dibromopyridine as presented in SCHEME 2 below. Addition of a metalated bromopyridine, prepared by transmetalation of 2,5-dibromopyridine with a base such as n-butyllithium in a suitable solvent such as ether or THF, to III provides secondary alcohol VIII. Conversion of VIII into the corresponding secondary chloride or bromide IX is accomplished by reaction with an appropriate halogenating reagent, such as thionyl chloride or thionyl bromide, and an organic base, such as pyridine, diisopropylethylamine or triethylamine, in an organic solvent such as dichloromethane or toluene. Alkylation of the anion derived from deprotonation of an alkyl pyridylacetate with an appropriate base, such as lithium, sodium or potassium bis(trimethylsilyl)amide, with the halide IX in an appropriate organic solvent such as THF and/or HMPA (hexamethylphosphoramide), provides the ester X. Ester X is decarboxylated to give the pyridine XI by first hydrolysing the ester X in the presence of aqueous hydroxide, such as sodium hydroxide, in a mixture of protic and aprotic organic solvents, such as methanol or ethanol and THF, followed by heating the carboxylic acid in an organic solvent such as dimethylsulfoxide. The bromopyridine XI was carbonylated undre a carbon monoxide atmosphere in the presence of a palladium II catalyst such as acetate, a ligand such as 1,1′-bis(diphenylphosphino)ferrocene and an organic base such as diisopropylethylamine or triethylamine, in a mixture of organic solvent such as methanol and DMF, to afford ester XII. Reaction of XII with an oxidizing agent, such as m-CPBA (meta-chloroperoxybenzoic acid) or MMPP (monoperoxyphthalic acid, magnesium salt) provides the N-oxides XIII. The tertiary alcohol XIV was prepared by the addition of an excess of an alkyl metal such as methyl magnesium bromide on the ester XIII at subambient temperature in an organic solvent such as ether, THF or dichloromethane. Pyridine-N-oxide XIV is rearranged to pyridone of Formula Ib by heating the N-oxide in the presence of an anhydride such as trifluoroacetic anhydride or acetic anhydride and alternatively by treatment with the same anhydrides with an organic base such as pyridine, diisopropylethylamine or triethylamine in an organic solvent such as THF or DMF.

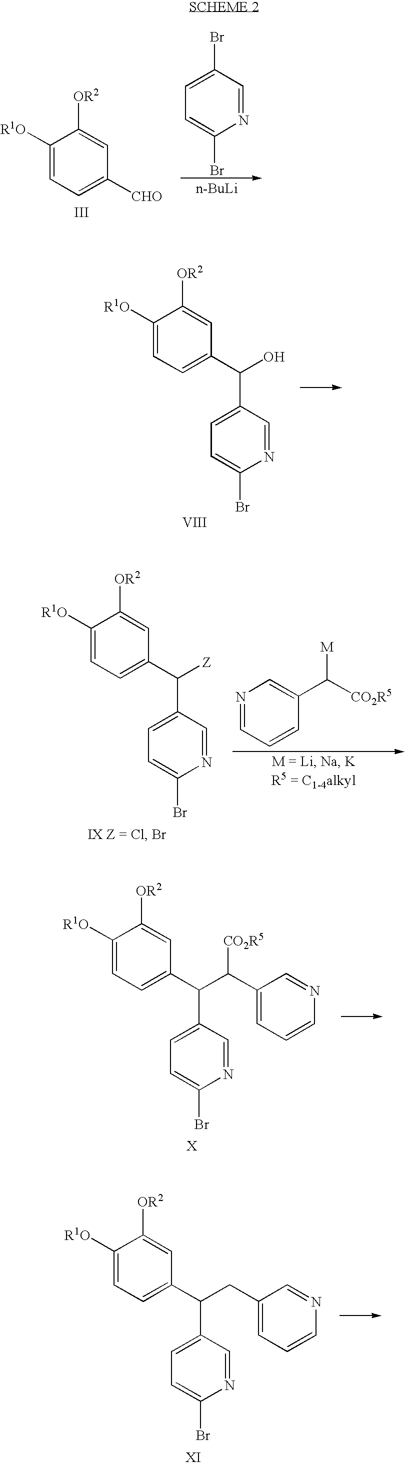

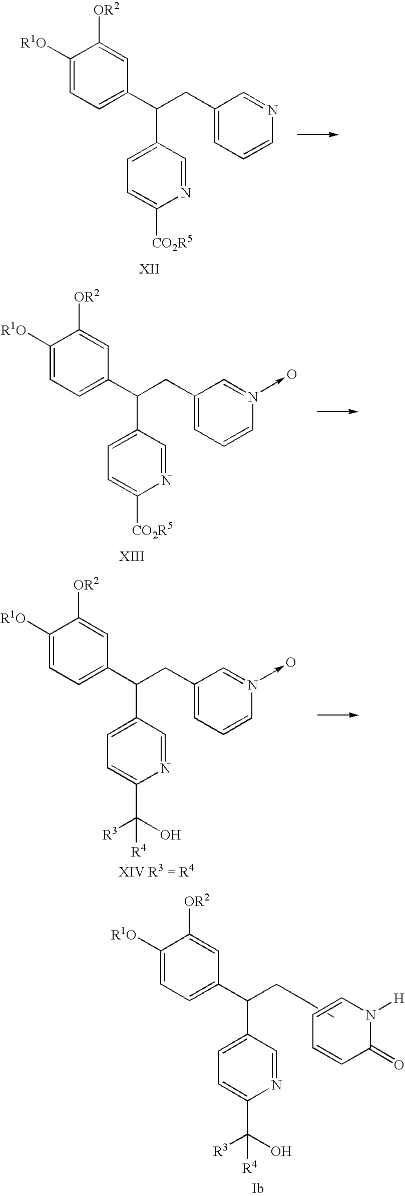

### EXAMPLES 1-9

Examples 1-9 are summarized in the table below:

| EX. | R¹ | R^{2b} | R³ | R⁴ | X | pyridone^{c} |
|---|---|---|---|---|---|---|
| 1 | CHF₂ | CHF₂ | CF₃ | CF₃ | Phenyl | 5-pyr |
| 2^{a} | CHF₂ | CHF₂ | CF₃ | CF₃ | Phenyl | 5-pyr |
| 3 | CHF₂ | CHF₂ | CH₃ | CH₃ | Phenyl | 5-pyr |
| 4 | CHF₂ | CHF₂ | CF₃ | CF₃ | Phenyl | 3-pyr |
| 5 | CHF₂ | CHF₂ | CH₃ | CH₃ | Pyridyl | 5-pyr |
| 6 | CHF₂ | c-Pr | CH₃ | CH₃ | Pyridyl | 5-pyr |
| 7^{a} | CHF₂ | c-Pr | CH₃ | CH₃ | Pyridyl | 5-pyr |
| 8 | CHF₂ | c-Pr | CF₃ | CF₃ | Thiazolyl | 5-pyr |
| 9 | CHF₂ | c-Pr | CF₃ | CF₃ | Thiazolyl | 3-pyr |

^{a}Example 2 and 7 are optically pure compounds.
^{b}“c-Pr” represents cyclopropyl.
^{c}“5-pyr” indicate that the 2-pyridone is linked to the ethyl residue at the 5 position, “3-pyr” indicate that the 2-pyridone is linked to the ethyl residue at the 3 position.

### EXAMPLES

All examples are mixtures of stereoisomers, either racemic mixtures (indicated as (±)) or racemic mixtures of diastereomers (indicated as (±/±)) unless stated otherwise. In those cases in which the stereoisomers have been separated, they are so indicated by Enantiomer 1, 2 etc. or Diastereomer 1, 2 etc.

### Example 1

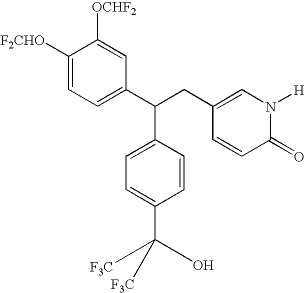

### (±)-5-{2-[3,4-BIS(DIFLUOROMETHOXY)PHENYL]-2-[4-(1,1,1,3,3,3-HEXAFLUORO-2-HYDROXYPROPAN-2-YL)PHENYL]ETHYL}2-PYRIDONE

EXAMPLE 1 was prepared by the following procedure:

### STEP 1: [3,4-Bis(difluoromethoxy)phenyl]-{4-[2-((2-trimethylsilylethoxy)methoxy)-1,1,1,1,3,3,3-hexafluoropropane-2-yl]phenyl}bromomethane

To a solution of 2.5 eq of pyridine in toluene was added at rt 1.2 eq of thionyl bromide followed by 1 eq of a 0.3M solution of [3,4-bis(difluoromethoxy)phenyl]-{4-[2-((2-trimethylsilylethoxy)methoxy)-1,1,1,3,3,3-hexafluoropropane-2-yl]phenyl methanol (US00/5710170A) in toluene. The mixture was stirred 40 min at rt and purified directly by chromatography eluting with toluene to afford the bromide.

### STEP 2: 3-{Carbethoxy-2-[3,4-bis(difluoromethoxy)phenyl]-2-[4-(2-((2-trimethylsilylethoxy)methoxy)-1,1,1,3,3,3-hexafluoropropan-2-yl)phenyl]ethyl}pyridine

To a 0° C. THF solution of ethyl 3-pyridylacetate (3 eq) was added 3 eq of HMPA followed by 3 eq of a 0.5M solution of KHMDS in toluene. This solution was stirred at 0° C. for 30 min, the ice bath was removed and 1 eq of a 0.3M solution of [3,4-bis(difluoromethoxy)phenyl]-{4-[2-((2-trimethylsilylethoxy)methoxy)-1,1,1,3,3,3-hexafluoropropane-2-yl] phenyl}bromomethane (EXAMPLE 1, STEP 1) in THF was added. The mixture was stirred at rt for 2 h and diluted with a 25% aqueous solution of NH₄OAc and ethyl acetate. The organic layer was washed with brine, dried over MgSO₄ and concentrated. Flash chromatography of the residue (silica gel; 40% EtOAc/hexane) provided the ester pyridine.

### STEP 3: 3-{2-[3,4-Bis(difluoromethoxy)phenyl]-2-[4-(1,1,1,3,3,3-hexafluoro-2-hydroxypropan-2-yl)phenyl]ethyl}pyridine

To a 0.1M solution of 3-{carbethoxy-2-[3,4-bis(difluoromethoxy)phenyl]-2-[4-(2-((2-trimethylsilylethoxy)methoxy)-1,1,1,3,3,3-hexafluoropropan-2-yl)phenyl]ethyl pyridine (EXAMPLE 1, STEP 2) in a mixture of THF/methanol/water (3:1:1), was added 3 eq of a 2M LiOH solution at rt. The mixture was stirred at 60° C. for 2.5 h, cooled down to rt, followed by the addition of 3.1 eq of a 1M HCl solution. After 10 min this mixture was concentrated under reduced pressure and diluted with a 25% aqueous solution of NH₄OAc and ethyl acetate. The organic layer was washed with brine, dried over MgSO₄ and concentrated. This carboxylic acid residue was dissolved in DMSO and stirred at 150° C. for 1 h. After cooling down to rt, the solution was diluted in ethyl acetate/water, the organic layer was washed twice with water, then with brine, dried over MgSO₄ and concentrated. Flash chromatography of the residue (silica gel; 50% EtOAc/hexane) provided the pyridine alcohol.

### STEP 4: 3-{2-[3,4-Bis(difluoromethoxy)phenyl]-2-[4-(1,1,1,3,3,3-hexafluoro-2-hydroxypropan-2-yl)phenyl]ethyl}pyridine-N-oxide

To a 0.08M solution of 3-{2-[3,4-bis(difluoromethoxy)phenyl]-2-[4-(1,1,1,3,3,3-hexafluoro-2-hydroxypropan-2-yl)phenyl]ethyl}pyridine (EXAMPLE 1, STEP 3) in a mixture of CH₂Cl₂/methanol (10:1), was added 1 eq of MMPP at rt. The mixture was stirred 4 h and purified directly by flash chromatography eluting with 10% of (10% NH₄OH/methanol) in CH₂Cl₂ to afford the pyridine-N-oxide.

### STEP 5: (±)-5-{2-[3,4-Bis(difluoromethoxy)phenyl]-2-[4-(1,1,1,3,3,3-hexafluoro-2-hydroxypropan-2-yl)phenyl]ethyl}2-pyridone

A 0.04M solution of 3-{2-[3,4-bis(difluoromethoxy)phenyl]-2-[4-(1,1,1,3,3,3-hexafluoro-2-hydroxypropan-2-yl)phenyl]ethyl}pyridine-N-oxide (EXAMPLE 1, STEP 4) in acetic anhydride was stirred at 140° C. for 6 h and concentrated under reduced pressure. The residue was dissolved in a 0.04M solution of THF/methanol (3:1) followed by the addition of 10 eq of a 2M solution of NaOH. The mixture was stirred 4 h at rt and diluted with a 25% aqueous solution of NH₄OAc and ethyl acetate. The organic layer was washed with brine, dried over MgSO₄ and concentrated. Flash chromatography of the residue (silica gel; 100% ethyl acetate and 10% ethanol/ethyl acetate) provided the desired pyridone: ¹HNMR (400 MHz, acetone-d₆): δ 3.17-3.27 (m, 2H), 4.43 (t, 1H), 6.22 (d, 1H), 6.92 (t, 1H), 6.94 (t, 1H), 7.12 (s, 1H), 7.25 (d, 1H), 7.29-7.36 (m, 2H), 7.38 (s, 1H), 7.53 (d, 2H), 7.7 (d, 2H).

### Example 2

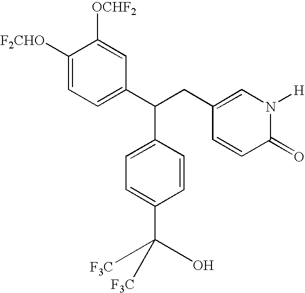

### CHIRAL 5-{2-[3,4-BIS(DIFLUOROMETHOXY)PHENYL]-2-[4-(1,1,1,3,3,3-HEXAFLUORO-2-HYDROXYPROPAN-2-YL)PHENYL]ETHYL}2-PYRIDONE

EXAMPLE 2 was prepared by the following procedure:

### STEP 1: Enantiomer (1) 3-{2-[3,4-bis(difluoromethoxy)phenyl]-2-[4-(1,1,1,3,3,3-hexafluoro-2-hydroxypropan-2-yl)phenyl]ethyl}pyridine

(±)3-{2-[3,4-Bis(difluoromethoxy)phenyl]-2-[4-(1,1,1,3,3,3-hexafluoro-2-hydroxypropan-2-yl)phenyl]ethyl}pyridine (EXAMPLE 1, STEP 3) was resolved using a preparatative Chiracel® AD HPLC column eluting with 10% ethanol/hexane at a flow rate of 70 mL/min. Enantiomer 1 and enantiomer 2 were collected at 25 min and 38 min respectively. Regardless of its absolute retention time, enantiomer 1 is defined has the fast eluting enantiomer under the conditions described.

### STEP 2: Enantiomer (1) 3-{2-[3,4-bis(difluoromethoxy)phenyl]-2-[4-(1,1,1,3,3,3-hexafluoro-2-hydroxypropan-2-yl)phenyl]ethyl}pyridine-N-oxide

The pyridine-N-oxide was obtained using the same procedure described for the STEP 4 of EXAMPLE 1.

### STEP 3: Enantiomer (1) 5-[2-[3,4-bis(difluoromethoxy)phenyl]-2-[4-(1,1,1,3,3,3-hexafluoro-2-hydroxypropan-2-yl)phenyl]ethyl}(2-pyridone)

To a 0.1M THF solution of enantiomer (1) 3-{2-[3,4-bis(difluoromethoxy)phenyl]-2-[4-(1,1,1,3,3,3-hexafluoro-2-hydroxypropan-2-yl)phenyl]ethyl}pyridine-N-oxide (EXAMPLE 2, STEP 2), was added 3 eq of triethylamine followed by 10 eq of trifluoroacetic anhydride at 0° C. The ice bath was removed and the solution was stirred at rt for 3 h. The reaction was quenched with a saturated aqueous solution of NaHCO₃ and diluted with ethyl acetate. The organic layer was washed with brine, dried over MgSO₄ and concentrated. Flash chromatography of the residue (silica gel; 10% ethanol/ethyl acetate) provided the desired pyridone: ¹HNMR (500M}z, acetone-d₆): δ 3.17-3.27 (m, 2H), 4.43 (t, 1H), 5.63 (s, 1H), 6.22 (d, 1H), 6.92 (t, 1H), 6.94 (t, 1H), 7.12 (s, 1H), 7.25 (d, 1H), 7.29-7.36 (m, 2H), 7.38 (s, 1H), 7.53 (d, 2H), 7.7 (d, 2H).

### Example 3

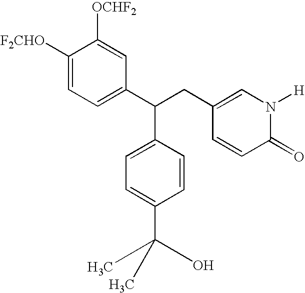

### (±)-5-{2-[3,4-BIS(DIFLUOROMETHOXY)PHENYL]-2-[4-(2-HYDROXYPROPAN-2-YL)PHENYL]ETHYL}2-PYRIDONE

EXAMPLE 3 was prepared by the following procedure:

### STEP 1: 2-(4-bromophenyl)-2-[(2-trimethylsilylethoxy)methoxy]propane

To a 0.2M DMF solution of 2-(4-bromophenyl)-2-propanol (JACS, 1971, 6877) was added 1.3 eq of sodium hydride at 0° C. in four portions. The ice bath was removed and the mixture was stirred 20 min at rt followed by the addition of 1.3 eq of SEMCl over 10 min. The reaction was stirred 2 h at rt and 1 h at 50° C. The reaction was quenched at rt with a saturated aqueous solution of NH₄Cl and diluted with ether and a 25% aqueous solution of NH₄OAc. The organic layer was washed 4 times with water, once with brine, dried over MgSO₄ and concentrated. Flash chromatography of the residue (silica gel; 5% ethyl acetate/hexane) provided the desired protected alcohol.

### STEP 2: [3,4-Bis(difluoromethoxy)phenyl]-{4-[2-((2-trimethylsilylethoxy)methoxy)propane-2-yl]phenyl}methanol

To 1.2 eq of a 0.2M THF solution of 2-(4-bromophenyl)-2-[(2-trimethylsilylethoxy)methoxy] propane (EXAMPLE 3, STEP 1) was added 1.2 eq of n-BuLi (1.6 M/hexanes) at −78° C. The solution was stirred 15 min at −78° C. followed by the addition of 1 eq of a 0.5M THF solution of 3,4-bis(difluoromethoxy) benzaldehyde (US00/5710170A). The resulting mixture was stirred 45 min at −78° C. and quenched with a saturated aqueous solution of NH₄Cl. The dry ice/acetone bath was removed and the reaction was diluted with ethyl acetate and a 25% aqueous solution of NH₄OAc. The organic layer was washed with brine, dried over MgSO₄ and concentrated. Flash chromatography of the residue (silica gel; 20% to 25% to 30% gradient of ethyl acetate/hexane) provided the desired secondary alcohol.

### STEP 3: {3,4-Bis(difluoromethoxy)phenyl]-{4-[2-((2-trimethylsilylethoxy)methoxy)propane-2-yl]phenyl}chloromethane

To a solution of 2.5 eq of pyridine in toluene was added at rt 1.2 eq of thionyl chloride followed by 1 eq of a 0.3M solution of [3,4-bis(difluoromethoxy)phenyl]-{4-[2-((2-trimethylsilylethoxy)methoxy)propane-2-yl]phenyl}methanol (EXAMPLE 3, STE[2) in toluene. The mixture was stirred 1 h at rt and purified directly by chromatography eluting with 20% ethyl acetate/toluene to afford the chloride.

### STEP 4: 3-{Carbethoxy-2-[3,4-bis(difluoromethoxy)phenyl]-2-[4-(2-((2-trimethylsilylethoxy)methoxy)-propan-2-yl)phenyl]ethyl}pyridine

To a 0° C. THF solution of ethyl 3-pyridylacetate (3 eq) was added 3 eq of HMPA followed by 3 eq of a 0.5M solution of KHMDS in toluene. This solution was stirred at 0° C. for 30 min, the ice bath was removed and 1 eq a 0.3M solution of [3,4-bis(difluoromethoxy)phenyl]-{4-[2-((2-trimethylsilylethoxy)methoxy)propane-2-yl]phenyl}chloromethane (EXAMPLE 3, STEP 3) in THF was added. The mixture was stirred at rt for 24 h, quenched with a saturated aqueous solution of NH₄Cl and diluted with a 25% aqueous solution of NH₄OAc and ethyl acetate. The organic layer was washed with brine, dried over MgSO₄ and concentrated. The residue was used directly in the next step without any purification.

### STEP 5: 3-{2-[3,4-Bis(difluoromethoxy)phenyl]-2-[4-(2-hydroxypropan-2-yl)phenyl]ethyl}pyridine

To a 0.1M solution of 3-{carbethoxy-2-[3,4-bis(difluoromethoxy)phenyl]-2-[4-(2-((2-trimethylsilylethoxy)methoxy)propan-2-yl)phenyl]ethyl}pyridine (EXAMPLE 3, STEP 4) in a mixture of THF/methanol/water (3:1:1), was added 3 eq of a 2M LiOH solution at rt. The mixture was stirred at 60° C. for 2.5 h, cooled down to rt, followed by the addition of 3.1 eq of a 1M HCl solution. After 10 min this mixture was concentrated under reduced pressure and diluted with a 25% aqueous solution of NH₄OAc and ethyl acetate. The organic layer was washed with brine, dried over MgSO₄ and concentrated. This carboxylic acid residue was dissolved in DMSO and stirred at 100° C. for 8 h and 114° C. for 3 h. After cooling down to rt, the solution was diluted in methylene chloride/water, the organic layer was washed with brine, dried over MgSO₄ and concentrated. This decarboxylated residue was dissolved in THF followed by the addition of 4.4 eq of a 1.0M solution of TBAF in THF and the reaction was refluxed overnight. The reaction was quenched at rt with a 25% aqueous solution of NH₄OAc and diluted with ethyl acetate. The organic layer was washed with brine, dried over MgSO₄ and concentrated. Flash chromatography of the residue (silica gel; gradient 50% EtOAc/hexane to 100% ethyl acetate) provided the pyridine alcohol.

### STEP 6: 3-{2-[3,4-Bis(difluoromethoxy)phenyl]-2-[4-(2-hydroxypropan-2-yl)phenyl]ethyl}pyridine-N-oxide

To a 0.08M solution of 3-{2-[3,4-bis(difluoromethoxy)phenyl]-2-[4-(2-hydroxypropan-2-yl)phenyl]ethyl}pyridine (EXAMPLE 3, STEP 5) in a mixture of CH₂Cl₂/methanol (10:1), was added 1 eq of MMPP at rt. The mixture was stirred 4 h and purified directly by flash chromatography eluting with 15% ethanol/CH₂Cl₂ to afford the pyridine-N-oxide.

### STEP 7: (±)-5-{2-[3,4-Bis(difluoromethoxy)phenyl]-2-[4-(2-hydroxypropan-2-yl)phenyl]ethyl}2-pyridone

To a 0.1M THF solution of 3-{2-[3,4-bis(difluoromethoxy)phenyl]-2-[4-(2-hydroxypropan-2-yl)phenyl]ethyl}pyridine-N-oxide (EXAMPLE 3, STEP 6), was added 3 eq of triethylamine followed by 10 eq of trifluoroacetic anhydride at 0° C. The ice bath was removed and the solution was stirred at rt for 4 h. The reaction was quenched with a saturated aqueous solution of NaHCO₃ and diluted with ethyl acetate. The organic layer was washed with brine, dried over MgSO₄ and concentrated. The residue was dissolved in THF/methanol/water (3:1:1) followed by the addition of 3 eq of a 1.7M solution of LiOH at rt. The solution was stirred 40 min, neutralized with 3.7 eq of a 2M HCl solution and diluted with a 25% aqueous solution of NH₄OAc and ethyl acetate. The organic layer was washed with brine, dried over MgSO₄ and concentrated. Flash chromatography of the residue (silica gel; gradient 5% to 10% ethanol/methylene chloride) provided the desired pyridone: ¹H NMR (acetone-D₆) δ 1.48 (s, 6H), 3.2 (d, 2H), 4.02, (bs, 1H), 4.32 (t, 1H), 6.28 (d, 1H), 6.92 (td, 2H), 7.15 (s, 1H), 7.2-7.4 (m, 7H), 7.45 (d, 2H).

### Example 4

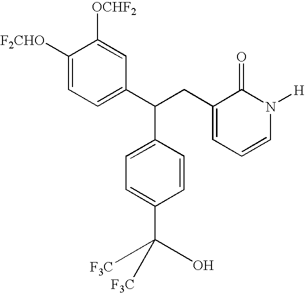

### (±)-3-{2-[3,4-BIS(DIFLUOROMETHOXY)PHENYL]-2-[4-(1,1,1,3,3,3-HEXAFLUORO-2-HYDROXYPROPAN-2-YL)PHENYL]ETHYL}2-PYRIDONE

EXAMPLE 4 was prepared by the following procedure:

### STEP 1: (O)-3-{2-[3,4-Bis(difluoromethoxy)phenyl]-2-[4-(1,1,1,3,3,3-hexafluoro-2-hydroxypropan-2-yl)phenyl]ethyl}2-pyridone

When 3-{2-[3,4-bis(difluoromethoxy)phenyl]-2-[4-(1,1,1,3,3,3-hexafluoro-2-hydroxypropan-2-yl)phenyl]ethyl}pyridine-N-oxide was treated under the conditions described at EXAMPLE 1, STEP 4, the regioisomeric 3-substituted 2-pyridone was also obtained: ¹HNMR (400 MHz, acetone-d₆): δ 3.19-3.31 (m, 2H), 4.72 (t, 1H), 5.97 (t, 1H), 6.90 (t, 1H), 6.96 (t, 1H), 7.12 (s, 1H), 7.20-7.30 (m, 3H), 7.37 (s, 1H), 7.53 (d, 2H), 7.7 (d, 2H).

### Example 5

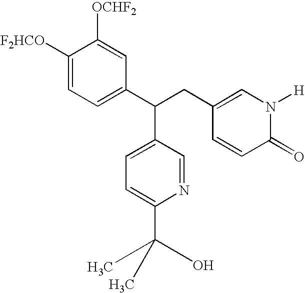

### (±)-5-{2-[3,4-BIS(DIFLUOROMETHOXY)PHENYL]-2-[2-(2-HYDROXYPROPAN-2-YL)5-PYRIDYL]ETHYL}2-PYRIDONE

EXAMPLE 5 was prepared by the following procedure:

### STEP 1: 3-{Carbethoxy-2-[3,4-bis(difluoromethoxy)phenyl]-2-(2-bromo-5-pyridyl) ethyl}pyridine

To a 0° C. THF solution of ethyl 3-pyridylacetate (1.9 eq) was added 1.9 eq of HMPA followed by 1.9 eq of a 0.5M solution of KHMDS in toluene. This solution was stirred at 0° C. for 30 min, the ice bath was removed and 1 eq a 0.5M solution of [3,4-bis(difluoromethoxy)phenyl]-(2-bromo-5-pyridyl) chloromethane in THF was added. The mixture was stirred at rt for 2 h, quenched with a saturated aqueous solution of NH₄Cl and diluted with ethyl acetate. The organic layer was washed with brine, dried over MgSO₄ and concentrated. The residue was used directly in the next step without any purification.

### STEP 2: 3-{2-[3,4-Bis(difluoromethoxy)phenyl]-2-(2-bromo-5-pyridyl)ethyl}pyridine

To a 0.1M solution of 3-{carbethoxy-2-[3,4-bis(difluoromethoxy)phenyl]-2-(2-bromo-5-pyridyl) ethyl}pyridine (EXAMPLE 5, STEP 1) in a mixture of THF/methanol/water (3:1:1), was added 3 eq of a 2M LiOH solution at rt. The mixture was stirred at 60° C. for 4 h and at rt overnight followed by the addition of 3 eq of a 1M HCl solution. After 10 min, this mixture was concentrated under reduced pressure and diluted with ethyl acetate. The aqueous layer was extracted 3 times with ethyl acetate in a range of pH from 4 to 7. The combined organic layers were washed with brine, dried over MgSO₄ and concentrated. This carboxylic acid residue was dissolved in DMSO and stirred at 150° C. for 3 h. After cooling down to rt, the solution was diluted in methylene chloride/water, the organic layer was washed twice with water, dried over MgSO₄ and concentrated. Flash chromatography of the residue provided the bromopyridine.

### STEP 3: 3-{2-[3,4-Bis(difluoromethoxy)phenyl]-2-(2-carbomethoxy-5-pyridyl)ethyl}pyridine

To a 0.3M solution of 3-{2-[3,4-bis(difluoromethoxy)phenyl]-2-(2-bromo-5-pyridyl)ethyl}pyridine (EXAMPLE 5, STEP 2) in DMF/methanol (1:1) was added 2 eq of triethylamine followed by 0.06 eq of 1,1′-bis(diphenylphosphino)ferrocene and 0.03 eq of palladium II acetate. This mixture was purge 3 times with carbon monoxide/vacuum and stirred overnight at 60° C. under a carbon monoxide atmosphere. The reaction was diluted with a 25% aqueous solution of NH₄OAc and ethyl acetate and the organic layer was washed 3 times with water, once with brine, dried over MgSO₄ and concentrated. Flash chromatography of the residue provided the methyl ester.

### STEP 4: 3-{2-[3,4-Bis(difluoromethoxy)phenyl]-2-(2-carbomethoxy-5-pyridyl)ethyl}pyridine-N-oxide

To a 0.05M solution 3-{2-[3,4-bis(difluoromethoxy)phenyl]-2-(2-carbomethoxy-5-pyridyl)ethyl}pyridine (EXAMPLE 5, STEP 3) in a mixture of CH₂Cl₂/methanol (10:1), was added 2 eq of MMPP at rt. The mixture was stirred overnight, an extra 0.4 eq of MMPP was added and the mixture was heated at 40° C. for 3 h and purified directly by flash chromatography eluting with 10% of (10% NH₄OH/methanol) in CH₂Cl₂ to afford the pyridine-N-oxide.

### STEP 5: 3-{2-[3,4-Bis(difluoromethoxy)phenyl]-2-[2-(2-hydroxypropan-2-yl)₅-pyridyl]ethyl} pyridine-N-oxide

To a 0.09M solution of 3-{2-[3,4-bis(difluoromethoxy)phenyl]-2-(2-carbomethoxy-5-pyridyl)ethyl}pyridine-N-oxide (EXAMPLE 5, STEP 4) in methylene chloride was added 5 eq of a 3.0M solution MeMgBr in ether at −78° C. The reaction was slowly warmed up to 0° C. over 1 h, monitered by TLC and quenched with a 25% aqueous solution of NH₄OAc. The mixture was diluted with ethyl acetate and the organic layer was washed with brine, dried over MgSO₄ and concentrated. This procedure was repeated again in order to consume all the ester starting material. Flash chromatography of the residue eluting with 10% of (10% N}40H/methanol) in CH₂Cl₂ provided the tertiary alcohol.

### STEP 6: (±)-5-{2-[3,4-Bis(difluoromethoxy)phenyl]-2-[2-(2-hydroxypropan-2-yl)₅-pyridyl]ethyl} 2-pyridone

To a 0.1M THF solution of 3-{2-[3,4-bis(difluoromethoxy)phenyl]-2-[2-(2-hydroxypropan-2-yl)5-pyridyl]ethyl} pyridine-N-oxide (EXAMPLE 5, STEP 5), was added 3 eq of triethylamine followed by 10 eq of trifluoroacetic anhydride at 0° C. The ice bath was removed and the solution was stirred at rt for 2 h. The reaction was quenched with a saturated aqueous solution of NaHCO₃ and diluted with ethyl acetate. The organic layer was washed with a saturated aqueous solution of NaHCO₃, brine, dried over MgSO₄ and concentrated. Flash chromatography of the residue eluting with 10% of (10% NH₄OH/methanol) in CH₂Cl₂ provided the pyridone trifluoroacetate intermediate. This ester was dissolved in THF/methanol/water (3:1:1) followed by the addition of 2 eq of a 2M solution of LiOH. The solution was stirred 1 h at rt and neutralized with 2 eq of a 2M solution of HCl. The reaction was then diluted with a 25% aqueous solution of NH₄OAc and ethyl acetate, the organic layer was washed with brine, dried over MgSO₄ and concentrated. Flash chromatography of the residue eluting with 15% ethanol/CH₂Cl₂ provided the pyridone: ¹HNMR (500 MHz, acetone-d₆): δ 1.44 (s, 6H), 3.17-3.28 (m, 2H), 4.44 (t, 1H), 4.60 (s, 1H), 6.24 (d, 1H), 6.93 (t, 1H), 6.96 (t, 1H), 7.11 (s, 1H), 7.28 (d, 1H), 7.33-7.37 (m, 2H), 7.41 (s, 1H), 7.57 (d, 1H) 7.80 (dd, 1H), 8.48 (s, 1H), 10.52 (s, 1H).

### Example 6

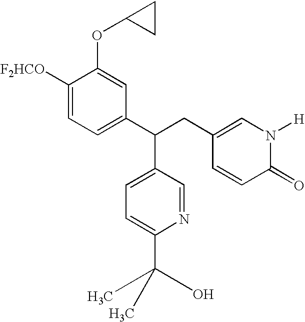

### (±)-5-{2-(3-CYCLOPROPYLOXY-4-DIFLUOROMETHOXYPHENYL)-2-[2-(2-HYDROXYPROPAN-2-YL)5-PYRIDYL]ETHYL}2-PYRIDONE

EXAMPLE 6 was prepared by the following procedure:

### STEP 1: 2-(2-hydroxypropan-2-yl)-5-bromopyridine

To a −78° C. suspension of 2,5-dibromopyridine in toluene (0.2M) was added n-butyllithium (1.1 eq.) and the reaction mixture stirred at −78° C. for 30 min. Acetone (1.2 eq.) was then added, the mixture stirred at −78° C. for 40 min, then quenched with saturated NH₄Cl. The aqueous phase was extracted with ethyl acetate, the organic layer washed once with brine, dried over MgSO₄, filtered and concentrated. The crude material was purified by flash chromatography on silica gel (20% ethyl acetate in hexanes) to afford 2-(2-hydroxypropan-2-yl)-5-bromopyridine as a yellow oil.

### STEP 2: 2-{2-[(2-trimethylsilylethoxy)methoxy]propan-2-yl}5-bromopyridine

Sodium hydride (1.4 eq.) was added portion-wise to a 0° C. solution of 2-(2-hydroxypropan-2-yl)-5-bromopyridine (EXAMPLE 6, STEP 1) in DMF (0.6M). The reaction mixture was warmed to rt, stirred for 1 h, then cooled to 0° C. SEMCl (1.3 eq.) was added and the mixture warmed to rt. After 4.5 h, the mixture was cooled to 0° C., sodium hydride (1 eq.) was added, followed by SEMCl (0.75 eq.). The reaction mixture was warmed to rt, stirred overnight, then poured into water at 0° C. The aqueous phase was extracted with ethyl acetate, the organic layer washed with water (3×), then brine, dried over MgSO₄, filtered and concentrated. The crude material was purified by flash chromatography on silica gel (2% ethyl acetate in hexanes) to afford the SEM-protected bromopyridine as a colourless oil.

### STEP 3: (3-Cyclopropyloxy-4-difluoromethoxyphenyl)-{2-[2-((2-trimethylsilylethoxy)methoxy)propane-2-yl] 5-pyridyl}methanol

To a solution of 2-{2-[(2-trimethylsilylethoxy)methoxy]propan-2-yl}5-bromopyridine (EXAMPLE 6, STEP 2) (1.2 eq.) in tetrahydrofuran (0.3M) at −78° C. was added n-butyllithium (1.2 eq.) and the solution stirred at −78° C. for 30 min. To this solution was then added a −78° C. solution of 3-cyclopropyloxy-4-difluoromethoxybenzaldehyde (WO 01/70738) in tetrahydrofuran (1.5M). After 5.5 h at −78° C., the reaction mixture was quenched with 25% NH₄OAc, the aqueous phase extracted with ethyl acetate, the organic layer washed once with brine, dried over Na₂SO₄, filtered and concentrated. The crude material was purified by flash chromatography on silica gel (30-40% ethyl acetate in hexanes) to afford the desired alcohol.

### STEP 4: (3-Cyclopropyloxy-4-difluoromethoxyphenyl)-{2-[2-((2-trimethylsilylethoxy)methoxy)propane-2-yl]5-pyridyl}chloromethane

To a 0° C. solution of pyridine (2.4 eq.) in toluene (0.2M) was added thionyl chloride (1.2 eq.), followed by a solution of (3-cyclopropyloxy-4-difluoromethoxyphenyl)-{2-[2-((2-trimethylsilylethoxy)methoxy)propane-2-yl] 5-pyridyl}methanol (EXAMPLE 6, STEP 3) in toluene (1M) after 5 min. The reaction mixture was warmed to rt, stirred for 1 h, then purified directly by flash chromatography on silica gel (20% ethyl acetate in hexanes) to afford the desired chloride as a yellow oil.

### STEP 5: 3-{Carbethoxy-2-(3-cyclopropyloxy-4-difluoromethoxyphenyl)-2-[2-(2-((2-trimethylsilylethoxy)methoxy)-propan-2-yl)5-pyridyl]ethyl}pyridine

HMPA (3 eq.) was added to a 0° C. solution of ethyl-3-pyridyl acetate (3 eq.) in tetrahydrofuran (0.3M), followed by addition of KHMDS (3 eq.). The mixture was stirred at 0° C. for 45 min, then a solution of (3-cyclopropyloxy-4-difluoromethoxyphenyl)-{2-[2-((2-trimethylsilylethoxy)methoxy)propane-2-yl] 5-pyridyl}chloromethane (EXAMPLE 6, STEP 4) in tetrahydrofuran (0.4M) was added. The reaction mixture was warmed to rt, stirred overnight, then quenched with saturated NH₄Cl. The aqueous phase was extracted with ethyl acetate, the organic layer washed once with brine, dried over Na₂SO₄, filtered and concentrated. The crude material was purified by flash chromatography on silica gel (50-70% ethyl acetate in hexanes) to afford the ester as a yellow oil.

### STEP 6: 3-{2-(3-Cyclopropyloxy-4-difluoromethoxyphenyl)-2-[2-(2-hydroxypropan-2-yl)5-pyridyl]ethyl}pyridine

Hydrolysis of 3-{carbethoxy-2-[(3-cyclopropyloxy-4-difluoromethoxy)phenyl]-2-[2-(2-((2-trimethylsilylethoxy)methoxy)-propan-2-yl)₅-pyridyl]ethyl}pyridine (EXAMPLE 6, STEP 5) was accomplished by treatment of a solution (0.09M) of ester in tetrahydrofuran:methanol:water (3:1:1) with lithium hydroxide (3 eq.), followed by heating at 60° C. for 2 h. The reaction mixture was cooled to rt, acidified with hydrochloric acid and concentrated. The resulting material was partitioned between ethyl acetate and water, the aqueous phase extracted with ethyl acetate at pH 0, 4 and 7. The organic layer was washed with brine, dried over Na₂SO₄, filtered and concentrated to provide the acid. A solution of this acid in DMSO (0.08M) was heated at 150° C. overnight, then poured into water and extracted with methylene chloride. The organic layer was washed with brine, dried over Na₂SO₄, filtered and concentrated. The crude material was purified by flash chromatography on silica gel (50-100% ethyl acetate in hexanes) to afford the desired hydroxypyridine.

### STEP 7: 3-{2-(3-Cyclopropyloxy-4-difluoromethoxyphenyl)-2-[2-(2-hydroxypropan-2-yl)5-pyridyl]ethyl}pyridine-N-oxide

Magnesium monoperoxyphthalate hexahydrate (1.1 eq.) was added to a solution (0.04M) of 3-{2-(3-cyclopropyloxy-4-difluoromethoxyphenyl)-2-[2-(2-hydroxypropan-2-yl)5-pyridyl]ethyl}pyridine (EXAMPLE 6, STEP 6) in methylene chloride:methanol (10:1) and the mixture stirred for 45 min, then purified directly by flash chromatography on silica gel (10-50% ethanol in ethyl acetate) to afford the N-oxide as a white powder.

### STEP 8: (±)-5-{2-(3-Cyclopropyloxy-4-difluoromethoxyphenyl)-2-[2-(2-hydroxypropan-2-yl)5-pyridyl]ethyl}2-pyridone

To a 0.1M THF solution of 3-{2-(3-cyclopropyloxy-4-difluoromethoxyphenyl)-2-[2-(2-hydroxypropan-2-yl)5-pyridyl]ethyl}pyridine-N-oxide (EXAMPLE 6, STEP 7), was added 3 eq of triethylamine followed by 10 eq of trifluoroacetic anhydride at 0° C. The ice bath was was removed and the solution was stirred at rt overnight. The reaction was quenched with a saturated aqueous solution of NaHCO₃ and diluted with ethyl acetate. The organic layer was washed with brine, dried over Na₂SO₄ and concentrated. The residue was dissolved in THF/methanol/water (3:1:1) followed by the addition of 3 eq of a 1M solution of LiOH. The solution was stirred 1 h at rt and neutralized with 3 eq of a 1M solution of HCl. The reaction was then diluted with a 25% aqueous solution of NH₄OAc and ethyl acetate, the organic layer was washed with brine, dried over Na₂SO₄ and concentrated. Flash chromatography of the residue eluting with 10% to 20% ethanol/ethyl acetate provided the pyridone: ¹H NMR (acetone-D₆) δ 0.6-0.9 (m, 4H), 1.45 (s, 6H), 3.18-3.28 (m, 2H), 3.88-3.94 (m, 1H), 4.39 (t, 1H), 4.7 (bs, 1H), 6.3 (d, 1H), 6.75 (t, 1H), 6.97 (d, 1H), 7.07 (d, 1H), 7.15 (s, 1H), 7.4 (dd, 1H), 7.46 (s, 1H), 7.58 (d, 1H), 7.82 (dd, 1H), 8.5 (s, 1H).

### Example 7

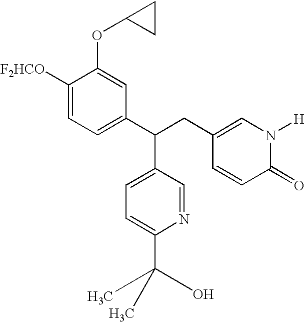

### CHIRAL-5-{2-(3-CYCLOPROPYLOXY-4-DIFLUOROMETHOXYPHENYL)-2-[2-(2-HYDROXYPROPAN-2-YL)5-PYRIDYL]ETHYL}2-PYRIDONE

EXAMPLE 7 was prepared by the following procedure:

### STEP 1: Enantiomer (1) 3-{2-(3-cyclopropyloxy-4-difluoromethoxyphenyl)-2-[2-(2-hydroxypropan-2-yl)5-pyridyl]ethyl}pyridine-N-oxide

(±)-3-{2-(3-Cyclopropyloxy-4-difluoromethoxyphenyl)-2-[2-(2-hydroxypropan-2-yl)5-pyridyl]ethyl}pyridine-N-oxide (EXAMPLE 6, STEP 7) was resolved using a preparatative Chiracel® AD HPLC column eluting with 40% ethanol/hexane at a flow rate of 60 mL/min. Regardless of its absolute retention time, enantiomer (1) is defined has the fast eluting isomer and enantiomer (2) the slow eluting isomer under the conditions described.

### STEP 2: Chiral-5-{2-(3-cyclopropyloxy-4-difluoromethoxyphenyl)-2-[2-(2-hydroxypropan-2-yl)5-pyridyl]ethyl}2-pyridone

To a 0.1M THF solution of enantiomer (1) 3-{2-(3-cyclopropyloxy-4-difluoromethoxyphenyl)-2-[2-(2-hydroxypropan-2-yl)5-pyridyl]ethyl}pyridine-N-oxide (EXAMPLE 7, STEP 1), was added 2.5 eq of triethylamine followed by 5 eq of trifluoroacetic anhydride at 0° C. The ice bath was removed and the solution was stirred at room temperature for 3 h. The reaction was quenched with 2M NaOH and stirred overnight at room temperature. The reaction was diluted with a 25% aqueous solution of NH₄OAc and ethyl acetate and the organic layer was washed with brine, dried over Na₂SO₄ and concentrated. This procedure has to be repeated again in order to consume all the starting material (3 eq of triethylamine and 10 eq of trifluoroacetic anhydride were used). Flash chromatography of the residue eluting with 7% to 10% ethanol/methylene chloride provided the pyridone: ¹H NMR (acetone-D₆) 60.64-0.85 (m, 4H), 1.44 (s, 6H), 3.16-3.26 (m, 2H), 3.87-3.93 (m, 1H), 4.36 (t, 1H), 4.61 (s, 1H), 6.24 (d, 1H), 6.74 (t, 1H), 6.96 (dd, 1H), 7.04-7.12 (m, 2H), 7.35 (dd, 1H), 7.46 (s, 1H), 7.56 (d, 1H), 7.80 (dd, 1H), 8.49 (s, 1H), 10.44 (bs, 1H).

### Example 8

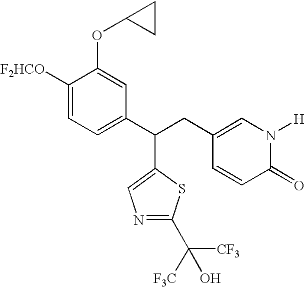

### CHIRAL-5-{2-(3-CYCLOPROPYLOXY-4-DIFLUOROMETHOXYPHENYL)-2-[2-(1-HYDROXY-1-TRIFLUOROMETHYL-2,2,2-TRIFLUOROETHYL)5-THIAZOLYL]ETHYL}2-PYRIDONE

EXAMPLE 8 was prepared by the following procedure:

### STEP 1: Chiral-5-{2-(3-cyclopropyloxy-4-difluoromethoxyphenyl)-2-[2-(1-hydroxy-1-trifluoromethyl-2,2,2-trifluoroethyl)5-thiazolyl]ethyl}2-pyridone

Triethylamine (3 eq) was added to a 0° C. solution of chiral-3-{2-(3-cyclopropyloxy-4-difluoromethoxyphenyl)-2-[2-(1-hydroxy-1-trifluoromethyl-2,2,2-trifluoroethyl)5-thiazolyl]ethyl} pyridine-N-oxide (WO 01/70738) in THF (0.05M), followed by the addition of trifluoroacetic anhydride (10 eq). The reaction mixture was warmed to rt, stirred overnight, then quenched with saturated NaHCO₃ and extracted with ethyl acetate. The organic layer was washed with brine, dried over NaSO₄, filtered and concentrated. The crude material was dissolved in THF/methanol/water (3:1:1) and LiOH (1 N, 3 equivalents) was added. After 3 h at rt, the reaction mixture was neutralized with HCl (1N, 3 eq), the volatile material removed under reduced pressure, and the residue partitioned between 25% NH₄OAc and ethyl acetate. The aqueous phase was extracted with ethyl acetate, the organic layer washed with brine, dried over NaSO₄, filtered and concentrated. The crude material was purified by flash chromatography on silica gel, using a gradient elution of 0-5% ethanol in ethyl acetate, to afford the desired pyridone: ¹H NMR (acetone-D₆) δ 0.6-0.9 (m, 4H), 3.22 (dd, 1H), 3.30 (dd, 1H), 3.88-3.91 (m, 1H), 4.73 (t, 1H), 6.30 (d, 1H), 6.78 (t, 1H), 6.99 (d, 1H), 7.11 (d, 1H), 7.18 (s, 1H), 7.40 (d, 1H), 7.45 (s, 1H), 7.81 (s, 1H).

### Example 9

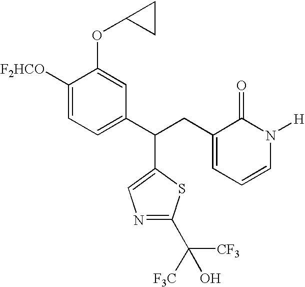

### CHIRAL-3-{2-(3-CYCLOPROPYLOXY-4-DIFLUOROMETHOXYPHENYL)-2-[2-(1-HYDROXY-1-TRIFLUOROMETHYL-2,2,2-TRIFLUOROETHYL)5-THIAZOLYL]ETHYL}2-PYRIDONE

EXAMPLE 9 was prepared by the following procedure:

### STEP 1: Chiral-3-{2-(3-cyclopropyloxy-4-difluoromethoxyphenyl)-2-[2-(1-hydroxy-1-trifluoromethyl-2,2,2-trifluoroethyl)5-thiazolyl]ethyl}2-pyridone

A solution of chiral-3-{2-(3-cyclopropyloxy-4-difluoromethoxyphenyl)-2-[2-(1-hydroxy-1-trifluoromethyl-2,2,2-trifluoroethyl)5-thiazolyl]ethyl} pyridine-N-oxide (WO 01/70738) in acetic anhydride (0.03M) was heated at 130° C. for 3.5 h, cooled to rt, diluted with 25% NH₄OAc and extracted with ethyl acetate. The organic layer was washed once with brine, dried over NaSO₄, filtered, concentrated and the residue placed under high vacuum for 0.5 h. The crude material was dissolved in THF/methanol/water (3:1:1) and LiOH (iN, 3 eq) was added. After 16 h at rt, the reaction mixture was neutralized with HCl (IN, 3 eq), the volatile material removed under reduced pressure, and the residue partitioned between saturated NaHCO₃ and ethyl acetate. The aqueous phase was extracted with ethyl acetate, the organic layer washed once with brine, dried over NaSO₄, filtered and concentrated. The crude material was purified by flash chromatography on silica gel, using a gradient elution of 2-5% ethanol in methylene chloride, to afford the pyridone: ¹H NMR (acetone-D₆) δ 0.59-0.85 (m, 4H), 3.22-3.33 (m, 2H), 3.88-3.91 (m, 1H), 5.04 (t, 1H), 6.03 (t, 1H), 6.77 (t, 1H), 6.95 (dd, 1H), 7.12 (dd, 2H), 7.32 (dd, 1H), 7.42 (d, 1H), 7.79 (s, 1H), 11.25 (bs, 1H).

1. A compound represented by Formula (I): 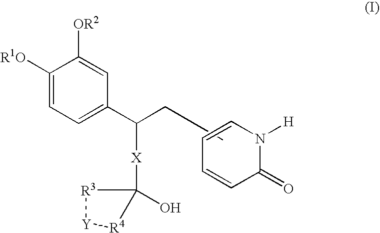
or a pharmaceutically acceptable salt thereof, wherein
X is phenyl, pyridinyl, thiazolyl, pyrimidinyl, pyridazinyl, furyl, thienyl, oxazolyl, isoxazolyl, isothiazolyl.
R¹ and R² are each independently —C₁₋₆alkyl, —C₃₋₆cycloalkyl, any of which optionally substituted with 1-6 independent halogen;
R³ and R⁴ are each independently —C₁₋₆alkyl, —C₃₋₆cycloalkyl, aryl, or heteroaryl. any of which optionally substituted with 1-6 independent halogen,
R³ and R⁴ are optionally connected by Y to form a ring, wherein Y is —C₁₋₆alkyl-.

2. A compound according to claim 1, or a pharmaceutically acceptable salt thereof, wherein X is phenyl, pyridinyl, or thiazolyl;

3. A compound according to claim 1, or a pharmaceutically acceptable salt thereof, wherein R³ and R⁴ are each independently —C₁₋₄alkyl optionally substituted with 1-6 independent halogen.

4. A compound according to claim 1, or a pharmaceutically acceptable salt thereof, wherein R³ and R⁴ are optionally connected by Y to form a ring, wherein Y is —C₁₋₄alkyl-.

5. The compound of claim 1, or a pharmaceutically acceptable salt thereof, wherein X is phenyl.

6. The compound of claim 1, or a pharmaceutically acceptable salt thereof, wherein X is phenyl; and R³ and R⁴ are each independently —C₁₋₄alkyl optionally substituted with 1-6 independent halogen.

7. The compound of claim 1, or a pharmaceutically acceptable salt thereof, wherein X is pyridinyl.

8. The compound of claim 1, or a pharmaceutically acceptable salt thereof, wherein X is pyridinyl; and R³ and R⁴ are each independently —C₁₋₄alkyl optionally substituted with 1-6 independent halogen.

9. The compound of claim 1, or a pharmaceutically acceptable salt thereof, wherein X is thiazolyl.

10. The compund of claim 1, or a pharmaceutically acceptable salt thereof, wherein X is thiazolyl; and R³ and R⁴ are each independently —C₁₋₄alkyl optionally substituted with 1-6 independent halogen.

11. The compound of claim 1, represented by 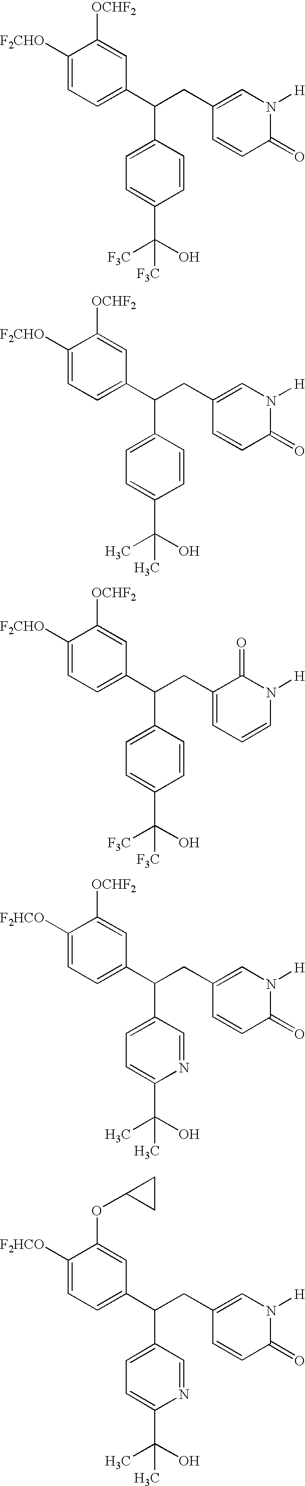 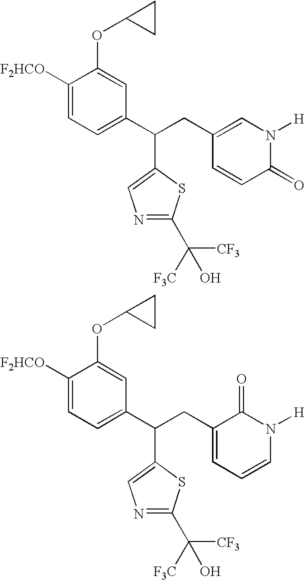
or a pharmaceutically acceptable salt thereof.

12. The compound according to claim 1, consisting of
(±)-5-{2-[3,4-Bis(difluoromethoxy)phenyl]-2-[4-(1,1,1,3,3,3-hexafluoro-2-hydroxypropan-2-yl)phenyl]ethyl}2-pyridone;
Chiral-5-{2-[3,4-bis(difluoromethoxy)phenyl]-2-[4-(1,1,1,3,3,3-hexafluoro-2-hydroxypropan-2-yl)phenyl]ethyl}2-pyridone;
(±)-5-{2-[3,4-Bis(difluoromethoxy)phenyl]-2-[4-(2-hydroxypropan-2-yl)phenyl]ethyl}2-pyridone;
(±)-3-{2-[3,4-Bis(difluoromethoxy)phenyl]-2-[4-(1,1,1,3,3,3-hexafluoro-2-hydroxypropan-2-yl)phenyl]ethyl}2-pyridone;
(±)-5-{2-[3,4-Bis(difluoromethoxy)phenyl]-2-[2-(2-hydroxypropan-2-yl)₅-pyridyl]ethyl}2-pyridone;
(±)-5-{2-(3-Cyclopropyloxy-4-difluoromethoxyphenyl)-2-[2-(2-hydroxypropan-2-yl)5-pyridyl]ethyl}2-pyridone;
Chiral-5-{2-(3-cyclopropyloxy-4-difluoromethoxyphenyl)-2-[2-(2-hydroxypropan-2-yl)5-pyridyl]ethyl}2-pyridone;
Chiral-5-{2-(3-cyclopropyloxy-4-difluoromethoxyphenyl)-2-[2-(1-hydroxy-1-trifluoromethyl-2,2,2-trifluoroethyl)5-thiazolyl]ethyl}2-pyridone;
Chiral-3-{2-(3-cyclopropyloxy-4-difluoromethoxyphenyl)-2-[2-(1-hydroxy-1-trifluoromethyl-2,2,2-trifluoroethyl)5-thiazolyl]ethyl}2-pyridone;
or a pharmaceutically acceptable salt thereof.

13. A pharmaceutical composition comprising a therapeutically effective amount of the compound according to claim 1 or a pharmaceutically acceptable salt thereof; and a pharmaceutically acceptable carrier.

14. The pharmaceutical composition according to claim 13, further comprising a Leukotriene receptor antagonist, a Leukotriene biosynthesis inhibitor, an M2/M3 antagonist, a corticosteroid, an H1 receptor antagonist or a beta 2 adrenoceptor agonist.

15. The pharmaceutical composition according to claim 13, further comprising a COX-2 selective inhibitor, a statin, or an NSAID.

16. A method of treatment or prevention of asthma; chronic bronchitis; chronic obstructive pulmonary disease; adult respiratory distress syndrome; infant respiratory distress syndrome; cough; chronic obstructive pulmonary disease in animals; adult respiratory distress syndrome; ulcerative colitis; Crohn's disease; hypersecretion of gastric acid; bacterial, fungal or viral induced sepsis or septic shock; endotoxic shock; laminitis or colic in horses; spinal cord trauma; head injury; neurogenic inflammation; pain; reperfusion injury of the brain; psoriatic arthritis; rheumatoid arthritis; ankylosing spondylitis; osteoarthritis; inflammation; or cytokine-mediated chronic tissue degeneration comprising the step of administering a therapeutically effective amount, or a prophylactically effective amount, of the compound according to claim 1 or a pharmaceutically acceptable salt thereof.

17. A method of treatment or prevention of allergic rhinitis, allergic conjunctivitis, eosinophilic granuloma, osteoporosis, arterial restenosis, atherosclerosis, reperfusion injury of the myocardium chronic glomerulonephritis, vernal conjunctivitis, cachexia, transplant rejection, or graft versus host disease, comprising the step of administering a therapeutically effective amount, or a prophylactically effective amount, of the compound according to claim 1 or a pharmaceutically acceptable salt thereof.

18. A method of treatment or prevention of depression, memory impairment, monopolar depression, Parkinson disease, Alzheimer's disease, acute and chronic multiple sclerosis, psoriasis, benign or malignant proliferative skin diseases, atopic dermatitis, urticaria, cancer, tumor growth or cancerous invasion of normal tissues, comprising the step of administering a therapeutically effective amount, or a prophylactically effective amount, of the compound according to claim 1 or a pharmaceutically acceptable salt thereof.

19-21. (canceled)
